(19) United States
(12) Patent Application Publication
(10) Pub. No.: US 2026/0224294 A1
(43) Pub. Date: Aug. 6, 2026

(54) TARGET-CENTERED VIRTUAL ELEMENT TO INDICATE HOLD TIME FOR MR REGISTRATION DURING SURGERY

(71) Applicant: Howmedica Osteonics Corp., Mahwah, NJ (US)

(72) Inventors: Damien Cariou, Loperhet (FR); Valentin Jouet, Brest (FR); Bryan Florentin Zago, Brest (FR); David José Thomas Boas, Brest (FR); Agathe Tréhin, Brest (FR); Steven Aurélien Vilmot, Saint-Renan (FR)

(21) Appl. No.: 19/160,284

(22) PCT Filed: Mar. 1, 2024
(86) PCT No.: PCT/US2024/018039

§ 371 Date: Aug. 27, 2025

Related U.S. Application Data
(60) Provisional application No. 63/487,747, filed on Mar. 1, 2023.

(51) Int. Cl. A61B 34/20; A61B 34/00; A61B 34/10; G06F 3/01

(52) U.S. Cl. CPC A61B 34/20; A61B 34/10; A61B 34/25; G06F 3/011; A61B 2034/102; A61B 2034/105; A61B 2034/252; A61B 2034/258

(57) ABSTRACT

A computing system includes a mixed reality (MR) visualization device and one or more processors implemented in circuitry may be configured to prompt a user wearing a visualization device to direct their gaze toward a physical registration region of a physical object that corresponds to a virtual registration region of a virtual model of the physical object; provide the user, via a user interface of the visualization device, a visual cue in a vicinity of the physical registration region; perform a registration operation to register the physical registration region with the virtual registration region; and while performing the registration operation, change the visual cue over time to indicate to the user a progress toward registration of the physical registration region with the virtual registration region.

This application claims priority to U.S. Provisional Patent Application No. 63/487,747, filed 1 Mar. 2023, the entire contents of which is incorporated herein by reference.

### BACKGROUND

Many types of surgical procedures involve inserting a surgical item into a bone of a patient. For example, a surgical procedure may include using a drill or impactor to create a hole, insert a pin, insert a screw, or insert a surgical nail into a bone of a patient. For instance, in one specific example, a surgeon may use a drill to insert a guide pin into a glenoid of a patient. Proper positioning of the surgical tool and insertion component may be a significant factor in the success of a surgical procedure. For instance, drilling a hole at an incorrect angle may lead to surgical complications.

### SUMMARY

As will be described in more detail below, when performing a surgical operation in an XR environment, a surgeon wearing an XR headset may periodically need to gaze at a specific location and/or hold instruments at certain locations while the XR system registers virtual models to real world objects. This registration process is computationally intensive and thus takes time to complete. While the XR system performs the registration, the surgeon needs to hold their head and/or their instruments steady otherwise the registration will be poor, resulting in blurry or misaligned scenes, or will require the registration to be re-performed, which adds time to the surgical operation. This disclosure describes techniques that may improve the user experience of a surgeon or other wearer of an XR headset by providing the wearer of the XR headset with visual cues that can result in quicker and better registrations between models of virtual objects and physical objects.

According to one example of the disclosure, a method for registration includes prompting a user wearing a visualization device to direct their gaze toward a physical registration region of a physical object that corresponds to a virtual registration region of a virtual model of the physical object; providing the user, via a user interface of the visualization device, a visual cue in a vicinity of the physical registration region; performing a registration operation to register the physical registration region with the virtual registration region; and while performing the registration operation, changing the visual cue over time to indicate to the user a progress toward registration of the physical registration region with the virtual registration region.

According to another example of the disclosure, a method for registration includes prompting a user wearing a visualization device to place a tip of a registration instrument at a location on a physical object in a physical registration region; after placement of the tip, identifying the registration instrument within the physical registration region; using a positioning of the registration instrument to identify the location on the physical object; based on the identified location, performing a registration operation to register a virtual model of the physical object with the physical object; providing the user, via a user interface of the visualization device, a visual cue in a vicinity of the physical registration region; and while performing the registration operation, changing the visual cue over time to indicate to the user a progress toward registration of the virtual model of the physical object with the physical object.

According to another example of the disclosure, a computing system includes a mixed reality (MR) visualization device and one or more processors implemented in circuitry configured to perform any of the techniques in this disclosure.

The details of one or more examples are set forth in the accompanying drawings and the description below. Other features, objects, and advantages will be apparent from the description, drawings, and claims.

### BRIEF DESCRIPTION OF THE DRAWINGS

FIG. 1 is a block diagram of a surgical system according to an example of this disclosure.

FIG. 2 is a schematic representation of a MR visualization device for use in the surgical system of FIG. 1, according to an example of this disclosure.

FIG. 3A illustrates example techniques for registering a 3-dimensional virtual bone model with an observed real bone structure of a patient during a joint repair surgery.

FIG. 3B illustrates example techniques for providing visual cues to a user during the registration of process FIG. 3A or during some other registration process.

FIG. 4 illustrates example techniques for providing visual cues to a user during a registration of process that utilizes a registration instrument.

FIG. 5A is a conceptual diagram illustrating an example MR scene with a visual cue in accordance with an example of this disclosure.

FIGS. 5B-5E show examples of alternate visual cues that may be displayed during a registration process in accordance with the techniques of this disclosure.

FIG. 6 is a conceptual diagram of virtual guidance that may be provided by the surgical assistance system, according to one or more examples of this disclosure.

FIG. 7 is a conceptual diagram of tools obscuring a portion of virtual guidance provided by an MR system.

FIGS. 8A-8F illustrate various views of various examples of a physical tracking tool, in accordance with one or more techniques of this disclosure.

FIG. 9 is a conceptual diagram illustrating an example MR scene in accordance with an example of this disclosure.

FIG. 10 is a flowchart illustrating an example operation of the surgical system corresponding to the MR scene of FIG. 9.

FIG. 11 is a conceptual diagram illustrating another example MR scene, in accordance with an example of this disclosure.

### DETAILED DESCRIPTION

Certain examples of this disclosure are described with reference to the accompanying drawings, wherein like reference numerals denote like elements. It should be understood, however, that the accompanying drawings illustrate only the various implementations described herein and are not meant to limit the scope of various technologies described herein. The drawings show and describe various examples of this disclosure.

In the following description, numerous details are set forth to provide an understanding of the present invention. However, it will be understood by those skilled in the art that the present invention may be practiced without these details and that numerous variations or modifications from the described examples may be possible.

A surgical procedure may involve inserting a surgical item into a bone of a patient. For example, the surgical procedure may involve using a drill or hammer to insert a surgical item, such as a drill bit, pin, screw, or nail into a bone of a patient. In such cases, because the surgical item is inserted into the bone of the patient, this disclosure may refer to such a surgical item as an insertable item.

Accordingly, this disclosure describes systems and methods associated with using mixed reality (MR) to assist with creation, implementation, verification, and/or modification of a surgical plan before and during a surgical procedure. Visualization tools other than or in addition to mixed reality visualization systems may be used in accordance with techniques of this disclosure. A surgical plan, e.g., as generated by the BLUEPRINT™ system or another surgical planning platform, may include information defining a variety of features of a surgical procedure, such as features of particular surgical procedure steps to be performed on a patient by a surgeon according to the surgical plan including, for example, bone or tissue preparation steps and/or steps for selection, modification and/or placement of implant components. Such information may include, in various examples, dimensions, shapes, angles, surface contours, and/or orientations of implant components to be selected or modified by surgeons, dimensions, shapes, angles, surface contours and/or orientations to be defined in bone or tissue by the surgeon in bone or tissue preparation steps, and/or positions, axes, planes, angle and/or entry points defining placement of implant components by the surgeon relative to patient bone or tissue. Information such as dimensions, shapes, angles, surface contours, and/or orientations of anatomical features of the patient may be derived from imaging (e.g., x-ray, CT, MRI, ultrasound or other images), direct observation, or other techniques.

In this disclosure, the term “mixed reality” (MR) refers to the presentation of virtual objects such that a user sees images that include both real, physical objects and virtual objects. Virtual objects may include text, 2-dimensional surfaces, 3-dimensional models, or other user-perceptible elements that are not actually present in the physical, real-world environment in which they are presented as coexisting. In addition, virtual objects described in various examples of this disclosure may include graphics, images, animations or videos, e.g., presented as 3D virtual objects or 2D virtual objects. Virtual objects may also be referred to as virtual elements. Such elements may or may not be analogs of real-world objects. In some examples, in mixed reality, a camera may capture images of the real world and modify the images to present virtual objects in the context of the real world. In such examples, the modified images may be displayed on a screen, which may be head-mounted, handheld, or otherwise viewable by a user. This type of mixed reality is increasingly common on smartphones, such as where a user can point a smartphone's camera at a sign written in a foreign language and see in the smartphone's screen a translation in the user's own language of the sign superimposed on the sign along with the rest of the scene captured by the camera. In some examples, in mixed reality, see-through (e.g., transparent) holographic lenses, which may be referred to as waveguides, may permit the user to view real-world objects, i.e., actual objects in a real-world environment, such as real anatomy, through the holographic lenses and also concurrently view virtual objects.

The Microsoft HOLOLENS™ headset, available from Microsoft Corporation of Redmond, Washington, is an example of a MR device that includes see-through holographic lenses, sometimes referred to as waveguides, that permit a user to view real-world objects through the lens and concurrently view projected 3D holographic objects. The Microsoft HOLOLENS™ headset, or similar waveguide-based visualization devices, are examples of an MR visualization device that may be used in accordance with some examples of this disclosure. Some holographic lenses may present holographic objects with some degree of transparency through see-through holographic lenses so that the user views real-world objects and virtual, holographic objects. In some examples, some holographic lenses may, at times, completely prevent the user from viewing real-world objects and instead may allow the user to view entirely virtual environments. The term mixed reality may also encompass scenarios where one or more users are able to perceive one or more virtual objects generated by holographic projection. In other words, “mixed reality” may encompass the case where a holographic projector generates holograms of elements that appear to a user to be present in the user's actual physical environment.

In some examples, in mixed reality, the positions of some or all presented virtual objects are related to positions of physical objects in the real world. For example, a virtual object may be tethered to a table in the real world, such that the user can see the virtual object when the user looks in the direction of the table but does not see the virtual object when the table is not in the user's field of view. In some examples, in mixed reality, the positions of some or all presented virtual objects are unrelated to positions of physical objects in the real world. For instance, a virtual item may always appear in the top right of the user's field of vision, regardless of where the user is looking.

Augmented reality (AR) is similar to MR in the presentation of both real-world and virtual elements, but AR generally refers to presentations that are mostly real, with a few virtual additions to “augment” the real-world presentation. For purposes of this disclosure, MR is considered to include AR. For example, in AR, parts of the user's physical environment that are in shadow can be selectively brightened without brightening other areas of the user's physical environment. This example is also an instance of MR in that the selectively-brightened areas may be considered virtual objects superimposed on the parts of the user's physical environment that are in shadow. Extended reality (XR) generically refers to any of AR, MR, or virtual reality (VR). It should be understood that the terms AR, MR, and VR can overlap with one another, and therefore, the use of a particular term to describe a technique or device should not be interpreted to exclude the use of other types of XR.

Accordingly, systems and methods are also described herein that can be incorporated into an intelligent surgical planning system, such as artificial intelligence systems to assist with planning, implants with embedded sensors (e.g., smart implants) to provide postoperative feedback for use by the healthcare provider and the artificial intelligence system, and mobile applications to monitor and provide information to the patient and the healthcare provider in real-time or near real-time.

Visualization tools are available that utilize patient image data to generate three-dimensional models of bone contours to facilitate preoperative planning for joint repairs and replacements. These tools allow surgeons to design and/or select surgical guides and implant components that closely match the patient's anatomy. These tools can improve surgical outcomes by customizing a surgical plan for each patient. An example of such a visualization tool for shoulder repairs is the BLUEPRINT™ system available from Stryker Corp. The BLUEPRINT™ system provides the surgeon with two-dimensional planar views of the bone repair region as well as a three-dimensional virtual model of the repair region. The surgeon can use the BLUEPRINT™ system to select, design or modify appropriate implant components, determine how best to position and orient the implant components and how to shape the surface of the bone to receive the components, and design, select or modify surgical guide tool(s) or instruments to carry out the surgical plan. The information generated by the BLUEPRINT™ system is compiled in a preoperative surgical plan for the patient that is stored in a database at an appropriate location (e.g., on a server in a wide area network, a local area network, or a global network) where it can be accessed by the surgeon or other care provider, including before and during the actual surgery.

As will be described in more detail below, when performing a surgical operation in an XR environment, a surgeon wearing an XR headset may periodically need to gaze at a specific location and/or hold instruments at certain locations while the XR system registers virtual models to real world objects. This registration process is computationally intensive and thus takes time to complete. While the XR system performs the registration, the surgeon needs to hold their head and/or their instruments steady otherwise the registration will be poor, resulting in blurry or misaligned scenes, or will require the registration to be re-performed, which adds time to the surgical operation. This disclosure describes techniques that may improve the user experience of a surgeon or other wearer of an XR headset by providing the wearer of the XR headset with visual cues that can result in quicker and better registrations between models of virtual objects and physical objects.

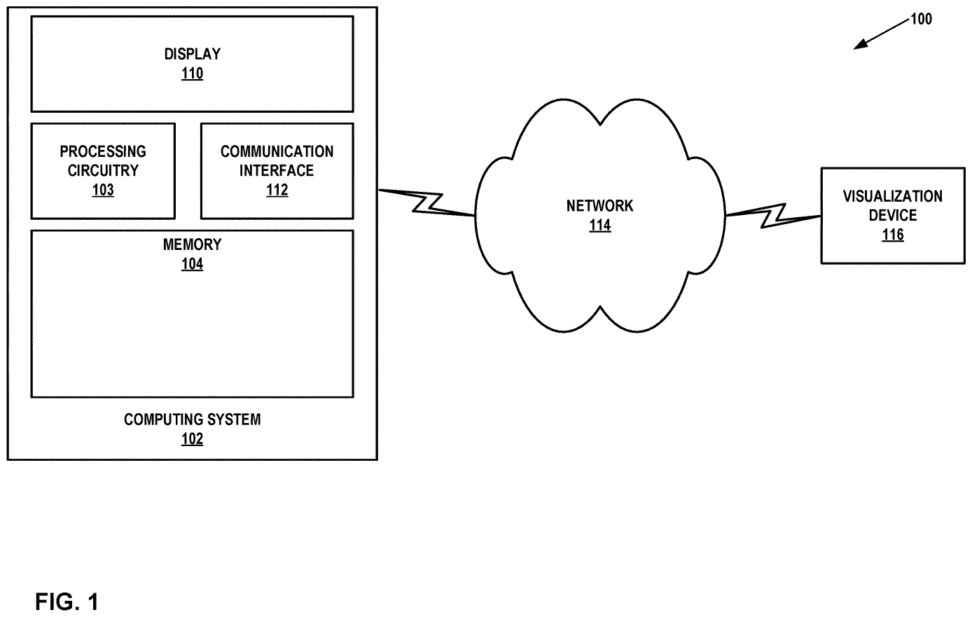

FIG. 1 is a block diagram illustrating an example surgical assistance system 100 that may be used to implement the techniques of this disclosure. FIG. 1 illustrates computing system 102, which is an example of one or more computing devices that are configured to perform one or more example techniques described in this disclosure.

Computing system 102 may include various types of computing devices, such as server computers, personal computers, smartphones, laptop computers, and other types of computing devices. In some examples, computing system 102 includes multiple computing devices that communicate with each other. In other examples, computing system 102 includes only a single computing device. Computing system 102 includes processing circuitry 103, memory 104, and a display 110. Display 110 is optional, such as in examples where computing system 102 is a server computer.

Examples of processing circuitry 103 include one or more microprocessors, digital signal processors (DSPs), application specific integrated circuits (ASICs), field programmable gate arrays (FPGAs), discrete logic, software, hardware, firmware or any combinations thereof. In general, processing circuitry 103 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. In some examples, processing circuitry 103 is dispersed among a plurality of computing devices in computing system 102. In some examples, processing circuitry 103 is contained within a single computing device of computing system 102.

Processing circuitry 103 may include arithmetic logic units (ALUs), elementary function units (EFUs), digital circuits, analog circuits, and/or programmable cores, formed from programmable circuits. In examples where the operations of processing circuitry 103 are performed using software executed by the programmable circuits, memory 104 may store the object code of the software that processing circuitry 103 receives and executes, or another memory within processing circuitry 103 (not shown) may store such instructions. Examples of the software include software designed for surgical planning, including image segmentation.

Memory 104 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 110 include a liquid crystal display (LCD), a plasma display, an organic light emitting diode (OLED) display, or another type of display device. In some examples, memory 104 may include multiple separate memory devices, such as multiple disk drives, memory modules, etc., that may be dispersed among multiple computing devices or contained within the same computing device.

Computing system 102 may include communication interface 112 that allows computing system 102 to output data and instructions to and receive data and instructions from visualization device 116 via network 114. For example, computing system 102 may output medical images, images of segmentation masks, and other information for display on visualization device 116.

Communication interface 112 may include hardware circuitry that enables computing system 102 to communicate (e.g., wirelessly or using wires) to other computing systems and devices, such as visualization device 116. Network 114 may include various types of communication networks including one or more wide-area networks, such as the Internet, local area networks, and so on. In some examples, network 114 may include wired and/or wireless communication links.

Visualization device 116 may utilize various visualization techniques to display image content to a surgeon. In some examples, visualization device 116 is a computer monitor or display screen. In some examples, visualization device 116 may be a mixed reality (MR) visualization device, VR visualization device, holographic projector, or other device for presenting XR visualizations. For instance, in some examples, visualization device 116 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses.

Visualization device 116 may use visualization tools that are available to utilize patient image data to generate three-dimensional models of bone contours, segmentation masks, or other data to facilitate preoperative planning. These tools may allow surgeons to design and/or select surgical guides and implant components that closely match the patient's anatomy. These tools can improve surgical outcomes by customizing a surgical plan for each patient. An example of such a visualization tool for shoulder repairs is the BLUEPRINT™ system available from Stryker Corp. The BLUEPRINT™ system provides the surgeon with two-dimensional planar views of the bone repair region as well as a three-dimensional virtual model of the repair region. The surgeon can use the BLUEPRINT™ system to select, design or modify appropriate implant components, determine how best to position and orient the implant components and how to shape the surface of the bone to receive the components, and design, select or modify surgical guide tool(s) or instruments to carry out the surgical plan. The information generated by the BLUEPRINT™ system may be compiled in a preoperative surgical plan for the patient that is stored in a database at an appropriate location (e.g., on a server in a wide area network, a local area network, or a global network) where the preoperative surgical plan can be accessed by the surgeon or other care provider, including before and during the actual surgery.

A surgical lifecycle begins with a preoperative phase. During the preoperative phase, a surgical plan is developed. The preoperative phase is followed by a manufacturing and delivery phase. During the manufacturing and delivery phase, patient-specific items, such as parts and equipment, needed for executing the surgical plan are manufactured and delivered to a surgical site. In some examples, it is unnecessary to manufacture patient-specific items in order to execute the surgical plan. An intraoperative phase follows the manufacturing and delivery phase. The surgical plan is executed during the intraoperative phase. In other words, one or more persons perform the surgery on the patient during the intraoperative phase. The visualization techniques of this disclosure may be performed primarily during the intraoperative phase. For example, surgical assistance system 100 may provide visual cues during the intraoperative phase to aid a surgeon or other user in performing registration. The intraoperative phase is followed by the postoperative phase (208). The postoperative phase includes activities occurring after the surgical plan is complete. For example, the patient may be monitored during the postoperative phase for complications.

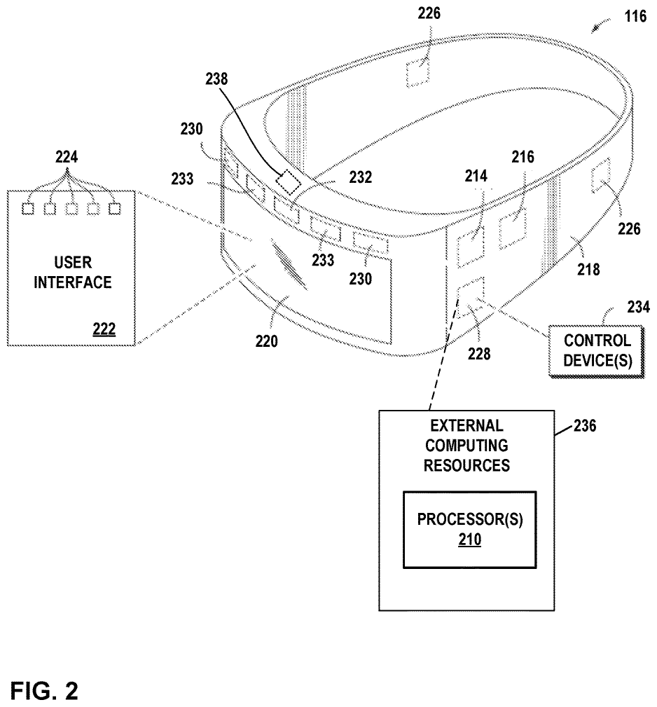

FIG. 2 is a schematic representation of visualization device 116 for use in an MR system, according to an example of this disclosure. As shown in the example of FIG. 2, visualization device 116 can include a variety of electronic components found in a computing system, including one or more processors 214 (e.g., microprocessors or other types of processing units) and memory 216 that may be mounted on or within a frame 218. Furthermore, in the example of FIG. 2, visualization device 116 may include a transparent screen 220 that is positioned at eye level when visualization device 116 is worn by a user. In some examples, screen 220 can include one or more liquid crystal displays (LCDs) or other types of display screens on which images are perceptible to a surgeon who is wearing or otherwise using visualization device 116 via screen 220. Other display examples include organic light emitting diode (OLED) displays. In some examples, visualization device 116 can operate to project 3D images onto the user's retinas using techniques known in the art.

In some examples, screen 220 may include see-through holographic lenses. sometimes referred to as waveguides, that permit a user to see real-world objects through (e.g., beyond) the lenses and also see holographic imagery projected into the lenses and onto the user's retinas by displays, such as liquid crystal on silicon (LCOS) display devices, which are sometimes referred to as light engines or projectors, operating as an example of a holographic projection system 238 within visualization device 116. In other words, visualization device 116 may include one or more see-through holographic lenses to present virtual images to a user. Hence, in some examples, visualization device 116 can operate to project 3D images onto the user's retinas via screen 220, e.g., formed by holographic lenses. In this manner, visualization device 116 may be configured to present a 3D virtual image to a user within a real-world view observed through screen 220, e.g., such that the virtual image appears to form part of the real-world environment. In some examples, visualization device 116 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses.

Although the example of FIG. 2 illustrates visualization device 116 as a head-wearable device, visualization device 116 may have other forms and form factors. For instance, in some examples, visualization device 116 may be a handheld smartphone or tablet.

Visualization device 116 can also generate a user interface (UI) 222 that is visible to the user, e.g., as holographic imagery projected into see-through holographic lenses as described above. For example, UI 222 can include a variety of selectable widgets 224 that allow the user to interact with a mixed reality (MR) system, such as computing system 102 of FIG. 2. Imagery presented by visualization device 116 may include, for example, one or more 3D virtual objects. Details of an example of UI 222 are described elsewhere in this disclosure. Visualization device 116 also can include a speaker or other sensory devices 226 that may be positioned adjacent the user's ears. Sensory devices 226 can convey audible information or other perceptible information (e.g., vibrations) to assist the user of visualization device 116.

Visualization device 116 can also include a transceiver 228 to connect visualization device 116 to external computing resources and/or to network 114 and/or to a computing cloud, such as via a wired communication protocol or a wireless protocol, e.g., Wi-Fi, Bluetooth, etc. Visualization device 116 also includes a variety of sensors to collect sensor data, such as one or more optical camera(s) 230 (or other optical sensors) and one or more depth camera(s) 232 (or other depth sensors), mounted to, on or within frame 218. In some examples, the optical sensor(s) 230 are operable to scan the geometry of the physical environment in which user of computing system 102 is located (e.g., an operating room) and collect two-dimensional (2D) optical image data (either monochrome or color). Depth sensor(s) 232 are operable to provide 3D image data, such as by employing time of flight, stereo or other known or future-developed techniques for determining depth and thereby generating image data in three dimensions. Other sensors can include motion sensors 233 (e.g., Inertial Mass Unit (IMU) sensors, accelerometers, etc.) to assist with tracking movement.

Computing system 102 processes the sensor data so that geometric, environmental, textural, etc. landmarks (e.g., corners, edges or other lines, walls, floors, objects) in the user's environment or “scene” can be defined and movements within the scene can be detected. As an example, the various types of sensor data can be combined or fused so that the user of visualization device 116 can perceive 3D images that can be positioned, or fixed and/or moved within the scene. When fixed in the scene, the user can walk around the 3D image, view the 3D image from different perspectives, and manipulate the 3D image within the scene using hand gestures, voice commands, gaze line (or direction) and/or other control inputs. As another example, the sensor data can be processed so that the user can position a 3D virtual object (e.g., a bone model) on an observed physical object in the scene (e.g., a surface, the patient's real bone, etc.) and/or orient the 3D virtual object with other virtual images displayed in the scene. As yet another example, the sensor data can be processed so that the user can position and fix a virtual representation of the surgical plan (or other widget, image or information) onto a surface, such as a wall of the operating room. Yet further, the sensor data can be used to recognize surgical instruments and the position and/or location of those instruments.

Visualization device 116 may include one or more processors 214 and memory 216, e.g., within frame 218 of the visualization device. In some examples, one or more external computing resources 236 process and store information, such as sensor data, instead of or in addition to in-frame processors, such as one or more processors 214 and memory 216. In this way, data processing and storage may be performed by one or more processors 214 and memory 216 within visualization device 116 and/or some of the processing and storage requirements may be offloaded from visualization device 116. Hence, in some examples, one or more processors that control the operation of visualization device 116 may be within the visualization device, e.g., by one or more processors 214. Alternatively, in some examples, at least one of the processors that controls the operation of visualization device 116 may be external to the visualization device, e.g., processing circuity 103 in computing system 102 or processing circuitry in external computing resources 236. Likewise, operation of visualization device 116 may, in some examples, be controlled in part by a combination of one or more processors 214 within the visualization device and one or more processors 210 external to the visualization device.

For instance, in some examples, processing of the sensor data can be performed by one or more processors 210. In some examples, one or more processors 214 and memory 216 mounted to frame 218 may provide sufficient computing resources to process the sensor data collected by cameras 230, 232 and motion sensors 233. In some examples, the sensor data can be processed using a Simultaneous Localization and Mapping (SLAM) algorithm, or other known or future-developed algorithm for processing and mapping 2D and 3D image data and tracking the position of visualization device 116 in the 3D scene. In some examples, image tracking may be performed using sensor processing and tracking functionality provided by the Microsoft HOLOLENS™ system, e.g., by one or more sensors and one or more processors 214 within a visualization device 116 substantially conforming to the Microsoft HOLOLENS™ device or a similar mixed reality (MR) visualization device.

In some examples, computing system 102 can also include user-operated control device(s) 234 that allow the user to operate computing system 102, use computing system 102 in spectator mode (either as master or observer), interact with UI 222 and/or otherwise provide commands or requests to external computing resources 236 or other systems connected to network 114. As examples, the control device(s) 234 can include a microphone, a touch pad, a control panel, a motion sensor or other types of control input devices with which the user can interact.

As discussed above, surgical lifecycle 300 may include a preoperative phase 302 (FIG. 2). One or more users may use surgical assistance system 100 in preoperative phase 302. For instance, surgical assistance system 100 may include a virtual planning system, e.g., implemented by computing system 102, to help the one or more users generate a virtual surgical plan that may be customized to an anatomy of interest of a particular patient. As described herein, the virtual surgical plan may include a 3-dimensional virtual model that corresponds to the anatomy of interest of the particular patient and a 3-dimensional model of one or more prosthetic components matched to the particular patient to repair the anatomy of interest or selected to repair the anatomy of interest. The virtual surgical plan also may include a 3-dimensional virtual model of guidance information to guide a surgeon in performing the surgical procedure, e.g., in preparing bone surfaces or tissue and placing implantable prosthetic hardware relative to such bone surfaces or tissue.

An orthopedic surgical system, such as surgical assistance system 100, may be configured to display virtual guidance including one or more virtual guides for performing work on a portion of a patient's anatomy. For instance, the visualization system may display virtual guidance that guides performance of a surgical step with the use of a physical tracking tool that attaches to a rotating tool. In some examples, a user such as a surgeon may view real-world objects in a real-world scene. The real-world scene may be in a real-world environment such as a surgical operating room. In this disclosure, the terms real and real-world may be used in a similar manner. The real-world objects viewed by the user in the real-world scene may include the patient's actual, real anatomy, such as an actual glenoid or humerus, exposed during surgery. The user may view the real-world objects via a see-through (e.g., transparent) screen, such as see-through holographic lenses, of a head-mounted MR visualization device, such as visualization device 116, and also see virtual guidance such as virtual MR objects that appear to be projected on the screen or within the real-world scene, such that the MR guidance object(s) appear to be part of the real-world scene, e.g., with the virtual objects appearing to the user to be integrated with the actual, real-world scene. For example, the virtual guidance may be projected on the screen of a MR visualization device, such as visualization device 116, such that the virtual guidance is overlaid on, and appears to be placed within, an actual, observed view of the patient's actual bone viewed by the surgeon through the transparent screen, e.g., through see-through holographic lenses. Hence, in this example, the virtual guidance may be a virtual 3D object that appears to be part of the real-world environment, along with actual, real-world objects.

A screen through which the surgeon views the actual, real anatomy and also observes the virtual objects, such as virtual anatomy and/or virtual surgical guidance, may include one or more see-through holographic lenses. The holographic lenses, sometimes referred to as “waveguides,” may permit the user to view real-world objects through the lenses and display projected holographic objects for viewing by the user. As discussed above, an example of a suitable head-mounted MR device for visualization device 116 is the Microsoft HOLOLENS™ headset, available from Microsoft Corporation, of Redmond, Washington, USA. The HOLOLENS™ headset includes see-through, holographic lenses, also referred to as waveguides, in which projected images are presented to a user.

The visualization device (e.g., visualization device 116) may be configured to display different types of virtual guidance. Examples of virtual guidance include, but are not limited to, a virtual point, a virtual axis, a virtual angle, a virtual path, a virtual plane, virtual reticle, and a virtual surface or contour. As discussed above, the device system (e.g., visualization device 116) may enable a user to directly view the patient's anatomy via a lens by which the virtual guides are displayed, e.g., projected. The virtual guidance may guide or assist various aspects of the surgery. For instance, a virtual guide may guide at least one of preparation of anatomy for attachment of the prosthetic or attachment of the prosthetic to the anatomy.

The visualization system may obtain parameters for the virtual guides from a virtual surgical plan, such as the virtual surgical plan described herein. Example parameters for the virtual guides include, but are not necessarily limited to, guide location, guide orientation, guide type, guide color, etc.

The visualization system may display a virtual guide in a manner in which the virtual guide appears to be overlaid on an actual, real object, within a real-world environment, e.g., by displaying the virtual guide(s) with actual, real-world objects (e.g., at least a portion of the patient's anatomy) viewed by the user through holographic lenses. For example, the virtual guidance may be 3D virtual objects that appear to reside within the real-world environment with the actual, real object.

The techniques of this disclosure are described below with respect to a shoulder arthroplasty surgical procedure. Examples of shoulder arthroplasties include, but are not limited to, reversed arthroplasty, augmented reverse arthroplasty, standard total shoulder arthroplasty, augmented total shoulder arthroplasty, hemiarthroplasty, revision procedures, and fracture repair. However, the techniques are not so limited, and the visualization system may be used to provide virtual guidance information, including virtual guides in any type of surgical procedure. Other example procedures in which an orthopedic surgical system, such as surgical assistance system 100, may be used to provide virtual guidance include, but are not limited to, other types of orthopedic surgeries; any type of procedure with the suffix “plasty,” “stomy,” “ectomy,” “clasia,” or “centesis,”; orthopedic surgeries for other joints, such as elbow, wrist, finger, hip, knee, ankle or toe, or any other orthopedic surgical procedure in which precision guidance is desirable. For instance, a visualization system may be used to provide virtual guidance for an ankle arthroplasty surgical procedure.

As discussed above, surgical assistance system 100 may receive a virtual surgical plan for attaching an implant to a patient and/or preparing bones, soft tissue or other anatomy of the patient to receive the implant. The virtual surgical plan may specify various surgical steps to be performed and various parameters for the surgical steps to be performed. As one example, the virtual surgical plan may specify a location on the patient's bone (e.g., glenoid, humerus, tibia, talus, etc.) for attachment of a guide pin. As another example, the virtual surgical plan may specify locations and/or orientations of one or more anchorage locations (e.g., screws, stems, pegs, keels, etc.).

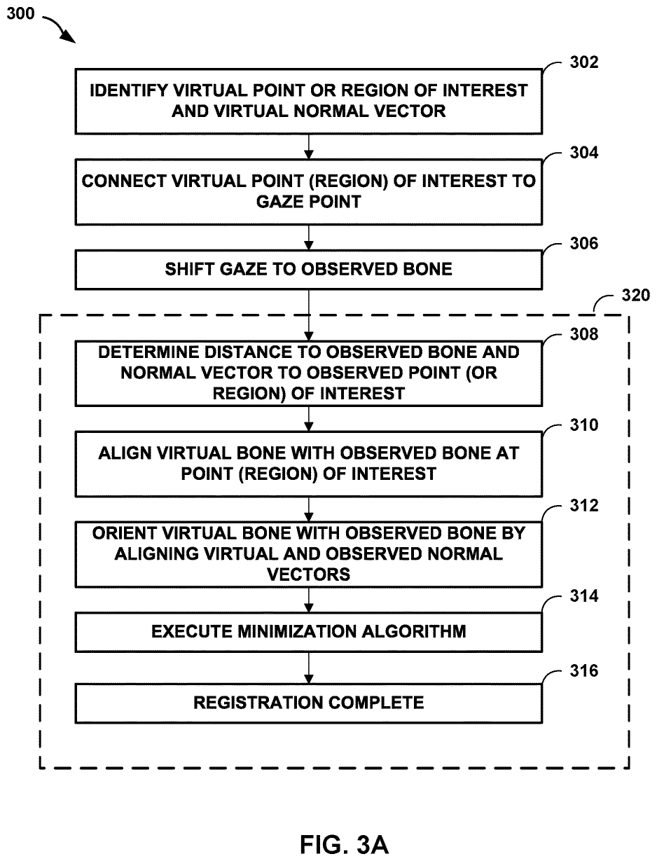

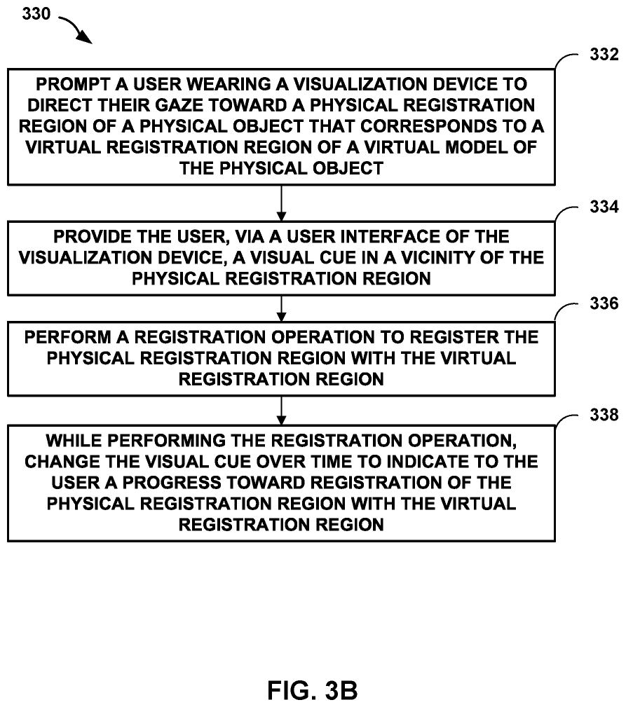

FIG. 3A illustrates an example of a process 300 for registering a 3D virtual bone model with a real observed bone structure of a patient. In other words, FIG. 3A is an example of a process flow, e.g., performed all or in part by visualization device 116, for registering a virtual bone model with an observed bone that is implemented in a mixed reality system, such as surgical assistance system 100. The process of FIG. 3A may be performed during the intraoperative phase of a surgical lifecycle.

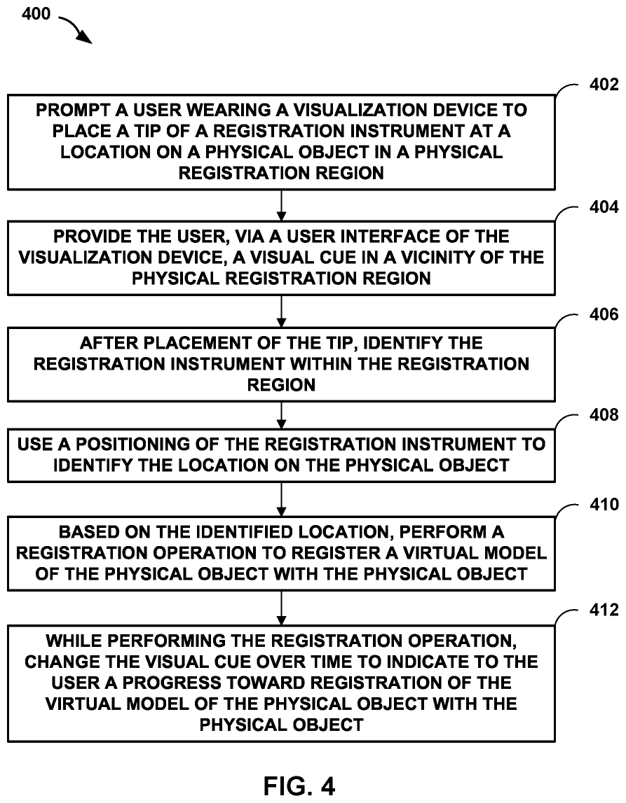

FIG. 3B illustrates an example process 330 for providing visual cues to a wearer of a visualization device while a portion of process 300 is being performed in conjunction with the techniques of this disclosure. Although described herein in conjunction with the registration techniques of process 300, process 330 may also be used in conjunction with other registration processes. FIG. 4 illustrates example techniques for providing visual cues to a wearer of a visualization device in conjunction with a registration process that utilizes a registration instrument. In some instances, the registration instrument may be referred to as a “digitizer” or “digitizer instrument.” The visual cues used in FIG. 4 may generally be the same visual cues used in FIG. 3B.

With further reference to FIG. 3A, the 3D virtual bone model may be a model of all or part of one or more bones. The process flow of FIG. 3A may be performed as part of a registration process. Such a registration process may occur multiple times during the intra-operative phase of a surgical procedure. The registration process may be carried out in two steps: initialization and optimization (e.g., minimization). During initialization, the user of surgical assistance system 100 uses the visualization device 116 in conjunction with information derived from a preoperative phase of the surgical lifecycle, the orientation of the user's head (which provides an indication of the direction of the user's eyes (referred to as “gaze” or “gaze line”), rotation of the user's head in multiple directions, sensor data collected by optical camera(s) 230, depth camera(s) 232, and motion sensors 233 (or other acquisitions sensors), and/or voice commands and/or hand gestures to visually achieve an approximate alignment of the 3D virtual bone model with observed bone structure. More particularly, at block 302, a point or region of interest on the surface of the virtual bone model and a virtual normal vector to the point (or region) of interest on the surface of the region are identified during the preoperative planning using surgical assistance system 100.

At block 304, surgical assistance system 100 connects the identified point (or region) of interest to the user's gaze point (e.g., a central point in the field of view of visualization device 116).

In the example of a shoulder arthroplasty procedure, the point of interest on the surface of the virtual bone model can be an approximate center of the virtual glenoid that can be determined by using a virtual planning system, such as the BLUEPRINT™ planning system. In some examples, the approximate center of the virtual glenoid can be determined using a barycenter find algorithm, with the assistance of machine learning algorithms or artificial intelligence systems, or using another type of algorithm. For other types of bone repair/replacement procedures, other points or regions of the bone can be identified and then connected to the user's gaze line or gaze point.

The ability to move and rotate the virtual bone model in space about the user's gaze point alone generally is not sufficient to orient virtual bone model with the observed bone. Thus, as part of the initialization procedure, surgical system also determines the distance between visualization device 116 and a point (or points) on the surface of the observed bone in the field of view of visualization device 116 and the orientation of that surface using sensor data collected from optical camera(s) 230, depth camera(s) 232, and motion sensors 233 (block 308). For example, a glenoid is a relatively simple surface because, locally, it can be approximated by a plane. Thus, the orientation of the glenoid surface can be approximated by determining a vector that is normal (i.e., perpendicular) to a point (e.g., a central point) on the surface. This normal vector is referred to herein as the “observed normal vector.” It should be understood, however, that other bones may have more complex surfaces, such as the humerus or knee. For these more complex cases, other surface descriptors may be used to determine orientation.

Regardless of the particular bone, distance information can be derived by surgical assistance system 100 from depth camera(s) 232 or other appropriately calibrated cameras. This distance information can be used to derive the geometric shape of the surface of an observed bone. That is, because depth camera(s) 232 provide distance data corresponding to any point in a field of view of depth camera(s) 232, the distance to the user's gaze point on the observed bone can be determined. With this information, the user can then move the 3D virtual bone model in space and approximately align it with the observed bone at a point or region of interest using the gaze point (block 310 in FIG. 3A). That is, when the user shifts gaze to the observed bone structure (block 306 in FIG. 3A), the virtual bone model (which is connected to the user's gaze line) moves with the user's gaze. The user can then the align 3D virtual bone model with observed bone structure by moving the user's head (and thus the gaze line), using hand gestures, using voice commands, and/or using a virtual interface to adjust the position of the virtual bone model. For instance, once the 3D virtual bone model is approximately aligned with the observed bone structure, the user may provide a voice command (e.g., “set”) that causes surgical assistance system 100 to capture the initial alignment. The orientation (“yaw” and “pitch”) of the 3D model can be adjusted by rotating the user's head, using hand gestures, using voice commands, and/or using a virtual interface which rotate the 3D virtual bone model about the user's gaze line so that an initial (or approximate) alignment of the virtual and observed objects can be achieved (block 312 in FIG. 3A). In this manner, the virtual bone model is oriented with the observed bone by aligning the virtual and observed normal vectors. Additional adjustments of the initial alignment can be performed as needed. For instance, after providing the voice command, the user may provide additional user input to adjust an orientation or a position of the virtual bone model relative to observed bone structure. This initial alignment process is performed intraoperatively (or in real time) so that the surgeon can approximately align the virtual and observed bones. In some examples, such as where the surgeon determines that the initial alignment is inadequate, the surgeon may provide user input (e.g., a voice command, such as “reset”) that causes surgical assistance system 100 to release the initial alignment.

At block 314 of FIG. 3A, when the user detects (e.g., sees) that an initial alignment of the 3D virtual bone model with observed bone structure has been achieved (at least approximately), the user can provide an audible or other perceptible indication to inform surgical assistance system 100 that a fine registration process (i.e., execution of an optimization (e.g., minimization) algorithm) can be started. For instance, the user may provide a voice command (e.g., “match”) that causes surgical assistance system 100 to execute a minimization algorithm to perform the fine registration process. The optimization process can employ any suitable optimization algorithm or process to perfect alignment of the virtual bone model with observed bone structure. The techniques of this disclosure are not limited to any particular registration or optimization algorithm or process and may, for example, be used with minimization algorithms, iterative closest point algorithms, deep-learning bases processes, or any other such algorithms or processes. At block 316 of FIG. 3A, upon completion of execution of the optimization algorithm, the registration procedure is complete.

While executing portions of process 300, such as portion 320 which includes the steps of blocks 308-316, it can be important for the user wearing visualization device 116 to hold their head relatively steady, e.g., without drift or jitter, for a period of time while surgical assistance system 100 executes the minimization algorithm. Additionally, if instruments are being registered, then it may also be important for the user wearing visualization device 116 to hold the instruments steady for a period of time while surgical assistance system 100 executes the minimization algorithm. This period of time will be referred to herein as a hold time and generally refers to the time it takes surgical assistance system 100 to perform a portion, e.g., portion 320, of registration process 300. For some registration processes, the hold time may be as much as 10 seconds. It should be understood that some steps of process 300 occur before, and possibly after, the hold time, and thus, the hold time represents only a portion of the time needed to perform process 300. The portion of the registration process performed during the hold time may, for example, include the minimization algorithm described with respect to block 314 in addition to other portions of process 300. If the wearer of visualization device 116 does not hold their head relatively steady during the hold time, then surgical assistance system 100 may obtain a relatively less accurate registration that results in image degradation such as blurring or fail to obtain an adequate registration altogether.

To improve the ability of a wearer of visualization device 116 to hold their head relatively steady during the hold time, this disclosure describes techniques for configuring visualization device 116 to output, via UI 222, one or more visual cues indicating to the user a progress toward registration of the first physical registration region with the first virtual registration region. The visual cue may, for example, be in the vicinity of the region of interest without obstructing the ability of a wearer of visualization device 116 to observe the region of interest. The visual cue may, for example, be an icon that fully or partially surrounds the center of a region of interest without obstructing the ability of a wearer of visualization device 116 to observe the center of the region of interest.

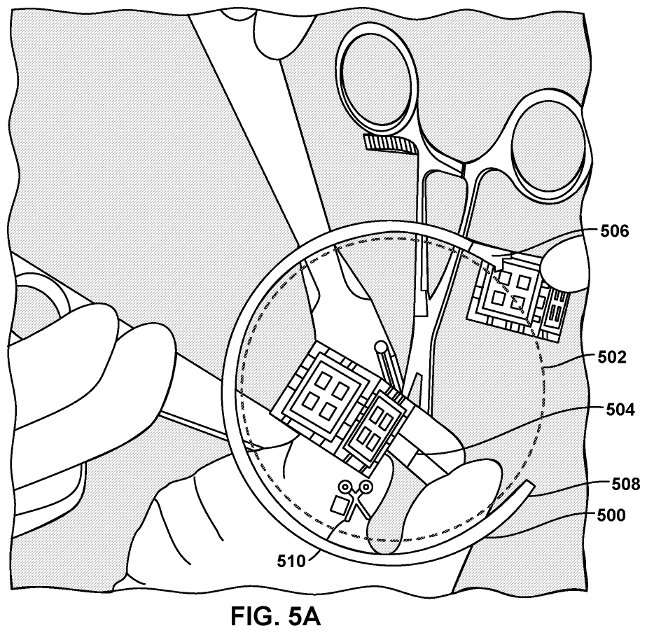

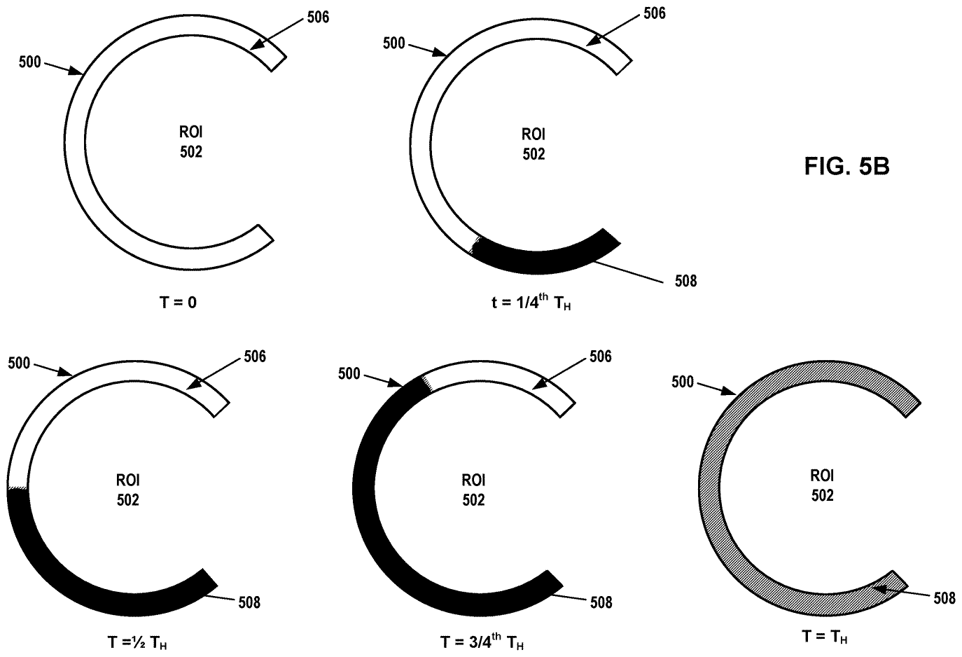

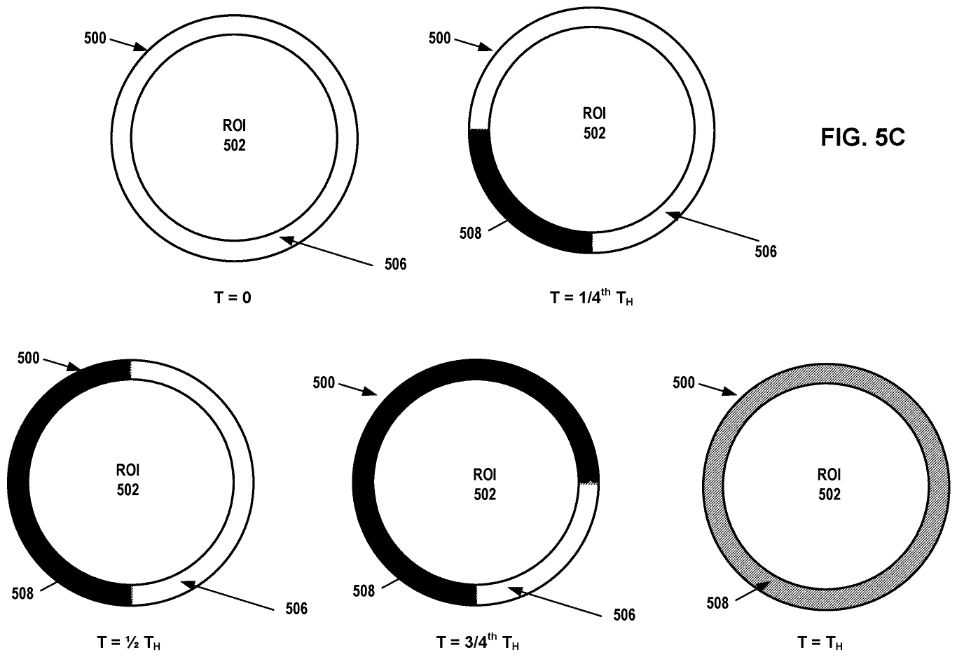

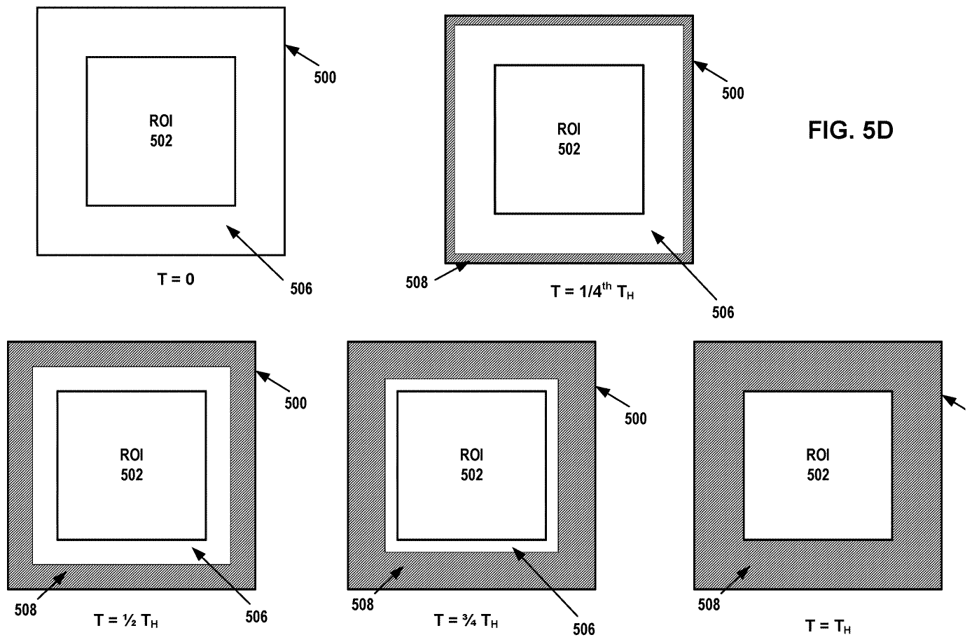

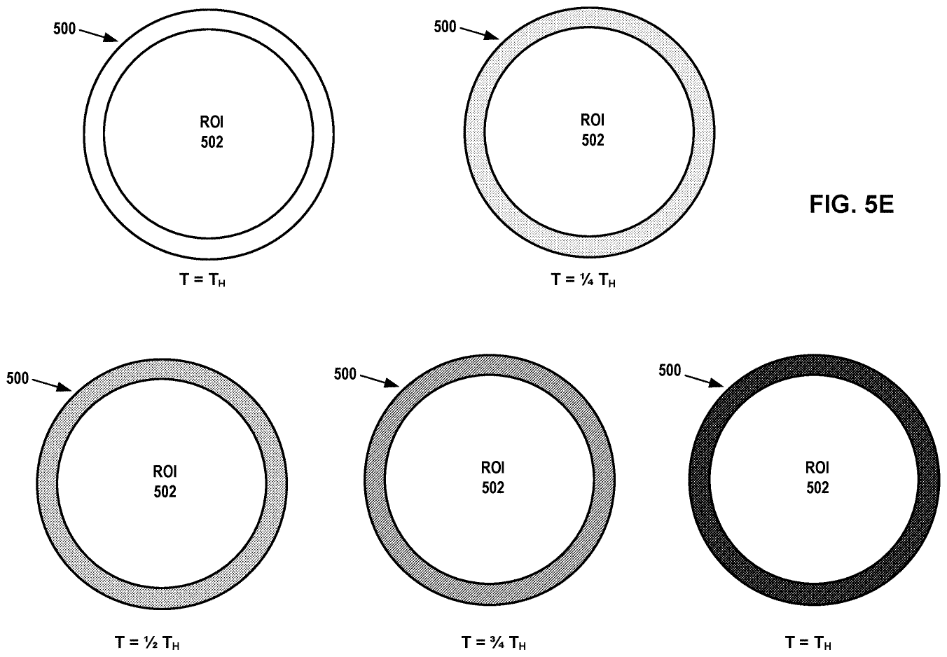

FIG. 3B shows an example process 330 for providing visual cues, such as those discussed below with respect to FIGS. 5A-5E. Surgical assistance system 100 may, for example, perform process 330 while simultaneously performing blocks 308-316 of FIG. 3A. In the example of FIG. 3B, surgical assistance system 100 may prompt a user wearing visualization device 116 to direct their gaze toward a physical registration region of a physical object that corresponds to a virtual registration region of a virtual model of the physical object (332). Surgical assistance system 100 may, for example, provide this prompt prior to performing the operation of block 306 in FIG. 3A. While performing the operations of boxes 308-316, surgical assistance system 100 may provide the user, via UI 222, a visual cue in a vicinity of the physical registration region (334). Surgical assistance system 100 may perform a registration operation (e.g., blocks 308-316 of FIG. 3A) to register the physical registration region with the virtual registration region (336). While performing the registration operation, surgical assistance system may change the visual cue over time to indicate to the user a progress toward registration of the physical registration region with the virtual registration region (338). Surgical assistance system 100 may change the visual cue to indicate to the user that registration of the physical registration region with the virtual registration region is complete in concurrence with block 316.

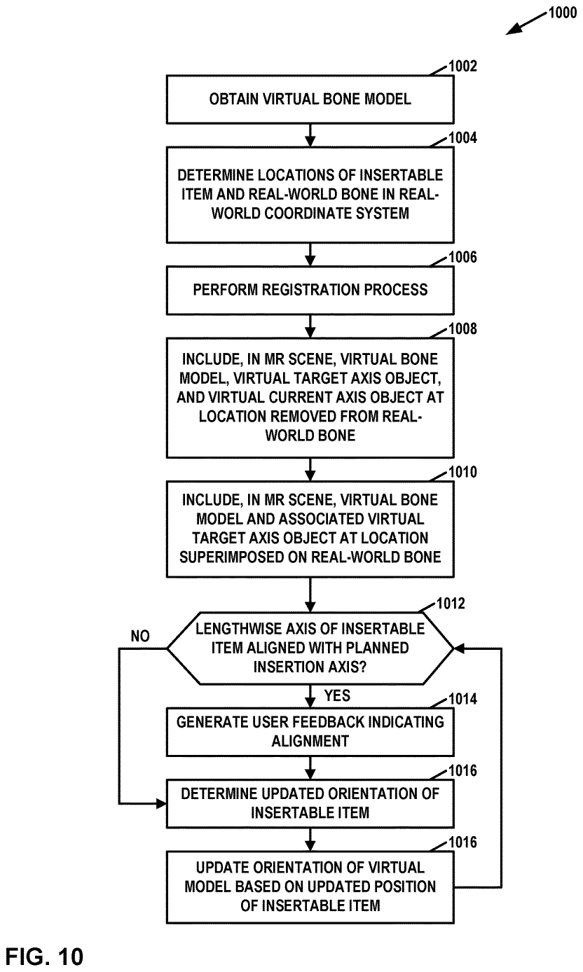

As mentioned above, process 330 may also be performed independent of process 300 or may be synchronized with process 300 in a different manner. Process 330 may, for example, also be performed in conjunction with process 1000 of FIG. 10, which will be described later in this disclosure.

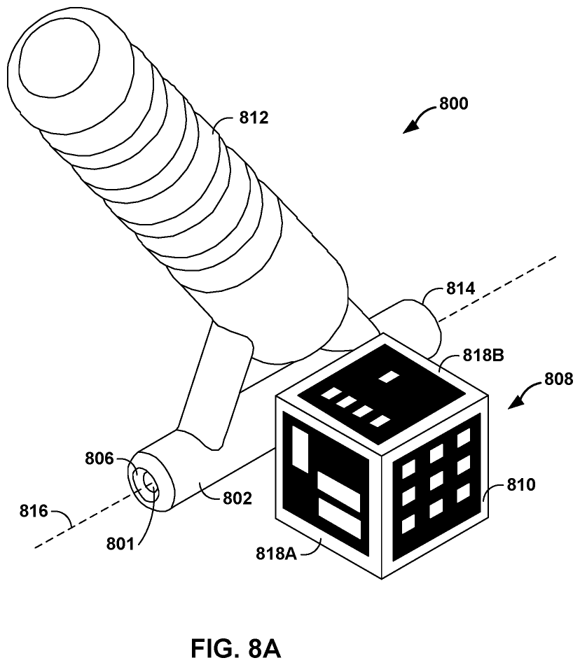

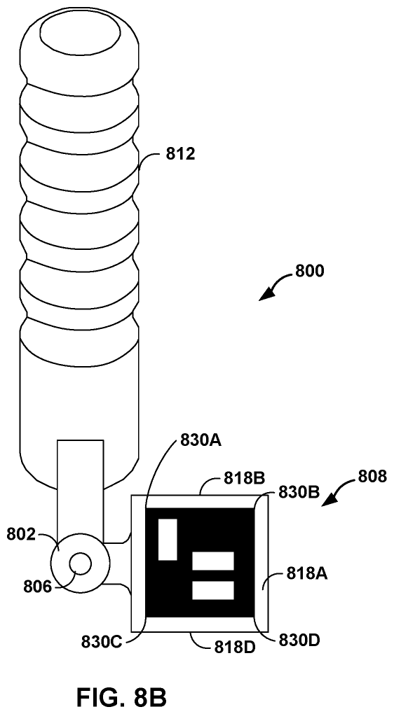

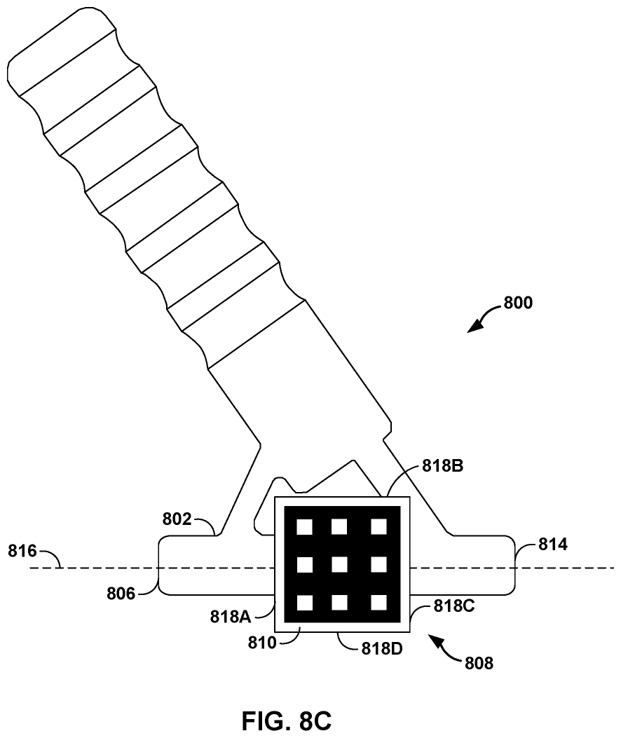

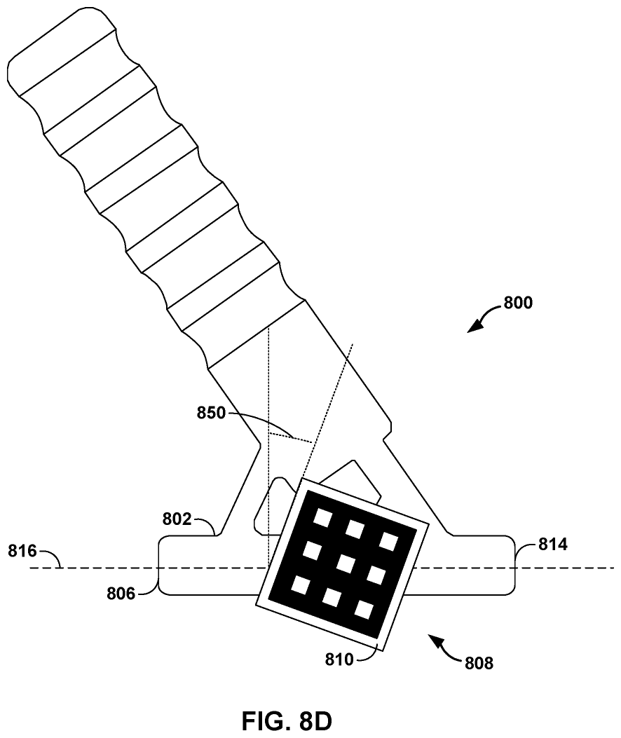

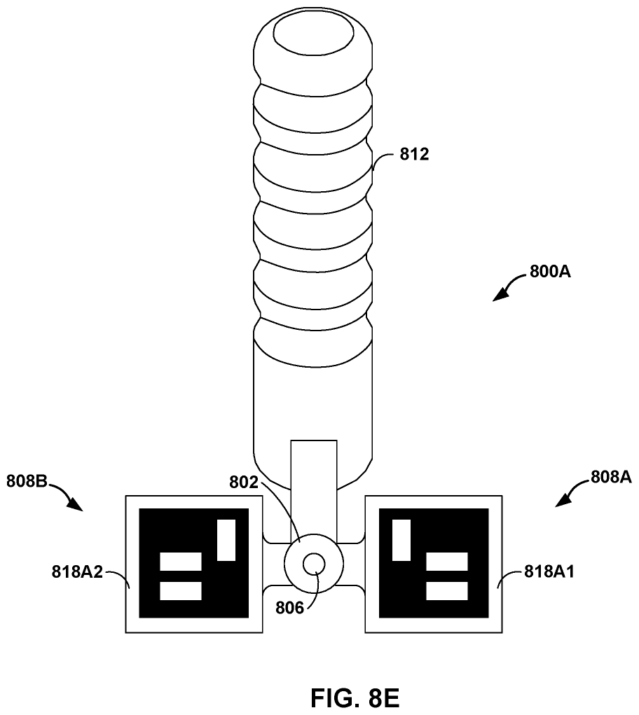

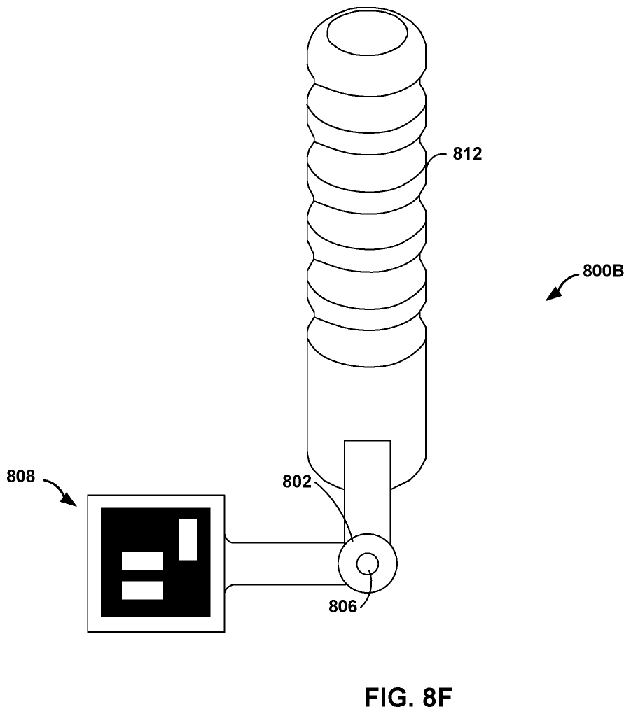

FIG. 4 illustrates example techniques for providing visual cues, such as those discussed below with respect to FIGS. 5A-5E, to a user during a registration process that utilizes a registration instrument. The visual cues used in FIG. 4 may be the same visual cues used in FIG. 3B, but FIG. 4A provides example techniques of those visual cues being used in a registration process that utilizes a registration instrument. Physical registration instrument 504 in FIG. 5A below is an example of such a registration instrument. The registration instrument may, for example, include a plurality of planar faces with each planar face having a different graphical pattern. As will be described in more detail with respect to the physical tracking tool of FIGS. 8A-8F, the planar faces may be used to determine a positioning of the registration instrument.

Surgical assistance system 100 prompts a user wearing a visualization device 116 to place a tip of a registration instrument at a location on a physical object in a physical registration region (402). The prompt may, for example, be a visual rendering of the virtual model with a point on the virtual model illuminated to identify the location on the physical object where the user is supposed to place the tip of the registration instrument. As one example, the physical object may be a patient's scapula or a portion thereof. To prompt the user, surgical assistance system 100 may render a visual representation of a virtual model of a scapula, with one point on the virtual model identified in some manner. The user may then touch the tip of the registration instrument to the point on the patient's scapula that corresponds to identified point on the visual rendering of the virtual model of the scapula. In some examples, surgical assistance system 100 may prompt the user to position the registration instrument at locations such as various landmark locations, such as landmark locations on the scapula. The landmark locations may include inferior, superior, anterior and posterior points on the rim of the glenoid fossa, a point on the tip of the coracoid process of the scapula, and so on. In some examples, surgical assistance system 100 may prompt the user to use the registration instrument to trace a curve on one or more surfaces of a bone. For instance, surgical assistance system 100 may prompt the user to use the registration instrument to trace a curve on the glenoid fossa, coracoid neck, and coracoid tip of a scapula.

Surgical assistance system 100 provides the user, via UI 222, a visual cue in a vicinity of the physical registration region (404). After placement of the tip of the registration instrument on the physical object (e.g., bone), surgical assistance system 100 identifies the registration instrument within the registration region (406). Surgical assistance system 100 uses a positioning of the registration instrument to identify the location on the physical object (408). Because one end of the registration instrument includes faces with different optical patterns and the registration instrument has a predefined length, surgical assistance system 100 may be able to determine the location of the tip of the registration instrument based on the locations and orientations of the optical patterns on the registration instrument. In some examples, surgical assistance system 100 may determine coordinates of the location on the physical object in an external coordinate system (e.g., a coordinate system representing positions of real-world objects, including the physical object).

Based on the identified location, surgical assistance system 100 performs a registration operation to register a virtual model with the physical object (410). For example, surgical assistance system 100 may determine a relationship between an internal virtual coordinate system (e.g., a coordinate system used in a surgical plan to define placement of surgical items, prostheses, etc.) and an external coordinate system (e.g., a coordinate system representing positions of real-world objects including the physical object). Surgical assistance system 100 may perform an iterative closest point (ICP) algorithm to determine the relationship between the points in the internal coordinate system and the external coordinate system. The result of the registration process may be a transform matrix that defines the relationship between the internal coordinate system and the external coordinate system.

While performing the registration operation, surgical assistance system 100 changes the visual cue over time to indicate to the user a progress toward registration of the virtual model of the physical object with the physical object (412). For instance, in an example where surgical assistance system 100 prompts the user to place the tip of the registration instrument at a particular landmark location, surgical assistance system 100 may change the visual cue may indicate how long the user should hold the registration instrument at the particular landmark location until surgical assistance system 100 has obtained sufficient video information to ascertain the location and orientation of the registration instrument and hence, the location of the particular landmark location. In an example where surgical assistance system 100 prompts the user to trace a curve on the physical object, the visual cue may indicate how long the user should continue tracing the curve on the physical object until the surgical assistance system 100 as obtained sufficient video information to ascertain the locations of a sufficient number of points in the physical object.

The ordering of steps shown in FIG. 4 represents only one example of the order in which the steps may be performed. For example, in some implementations, surgical assistance system 100 may not provide the user with the visual cue until after the user places the tip of the registration instrument at the location on the physical object. In some cases, a user may need to maintain a relatively steady head position while performing multiple iterations of steps 402-410. That is, the user may need to maintain a relatively steady head position, for example, while focused on the glenoid and while positioning the registration instrument at multiple positions on the glenoid surface. This process can take in excess of 30 seconds in some cases, and thus, can be challenging to users. The techniques of this disclosure may improve a user's ability to maintain the needed steady head position.

FIGS. 5A-5E provide examples of visual cues that may be used in conjunction with process 330 of FIG. 3B, process 400 of FIG. 4, or other such registration processes. FIG. 5A is a conceptual diagram illustrating an example MR scene with a visual cue in accordance with an example of this disclosure. FIG. 5A includes an example of visual cue 500, which surrounds region of interest 502. Visualization device 116 may output visual cue 500 to a wearer of visualization device 116 via UI 222. In the example of FIG. 5A, region of interest 502, which represents a physical registration region, includes a physical registration instrument 504 which is used to locate a point in the physical registration region. The point may, for example, be a point on a bone such as the scapula. As will be described in more detail later, surgical assistance system 100 may use the point identified by physical registration instrument 504 to aid in registering a virtual model of a physical object to the physical object in the physical registration region. In the example of FIG. 5A, the physical object is a scapula. In other examples, the regions of interest may not include an instrument, and surgical assistance system 100 may instead use other markers objects such as portions of a patient anatomy, implanted medical components, or other physical markers to perform registration. The techniques of this disclosure may also potentially be used in other contexts that require a user to hold their head steady or hold an instrument steady, while surgical assistance system 100 performs some sort of processing. Examples of such techniques might include intraoperative range of motion acquisition or camera calibration.

As can be seen in FIG. 5A, visual cue 500 surrounds, but does not substantially obstruct region of interest 502, such that the wearer of visualization device 116 can focus on region of interest 502. Moreover, by surrounding region of interest 502 in a manner such that a center of visual cue 500 approximately matches a center of region of interest 502, the wearer of visualization device 116 may be less inclined to allow their gaze to drift or to allow the positioning of instrument 504 to drift. In the example of FIG. 5A, the MR scene includes additional icons 510 inside of region of interest 502, but additional icons 510 are small relatively to the size of region of interest 502 are not centrally located within region of interest 502. Additional icons 510 are, however, close enough to a center of region of interest that a wearer of visualization device 116 can monitor additional icons 510 without provoking significant head movements. In one example, one of additional icons 510 may be an icon indicating how many faces of a plurality of planar faces of a physical tracking tool are visible, with for instance, red indicating 0, yellow indicating 1, and green indicating 2 or more. Registration and tracking in conjunction with planar faces of a physical tracking tool are described in more detail below.

As can be seen in FIG. 5A, visual cue 500 includes a first portion 506 which is a first color and a second portion 508 which is a second color. First portion 506 generally represents a percentage of the hold time that still remains, and second portion 508 generally represents a percentage of the hold time that has been completed. That is, the amount of visual cue 500 that is the first color, relative to the total size of visual cue 500, may generally be proportional to the amount of the hold time that has not yet been completed, and the amount of visual cue 500 that is the second color may generally be proportional to the amount of the hold time that has been completed.

Surgical assistance system 100 may be configured to size visual cue 500, such that visual cue 500 occupies space outside of region of interest 502 or such that visual cue 500 only occupies space in an outer region of visual cue and not in an inner portion of region of interest 502. Surgical assistance system 100 may additionally be configured to place visual cue 500 with an MR scene in a position such that a geometric center of visual cue 500 is located within region of interest 502. Referring back to block 402 of FIG. 4, surgical assistance system 100 may, for example, determine the size and location of the visual cue relative to the location on the physical object that that the tip of physical registration instrument 504 is to be placed. Surgical assistance system 100 may, for example, match a center (e.g., a geometric center or cent of curvature) of the visual cue to the location where the tip is to be placed. In other examples, surgical assistance system 100 may align the center of the visual cue with the location where the tip is to be placed, but with an offset so as to not obstruct other objects.

FIG. 5B shows an alternate representation of visual cue 500 surrounding region of interest (ROI) 502 at five different times (t in FIGS. 5B-5E) during the hold time. In the example of FIG. 5B, the hold time has duration T_{H}, and the five times are t=0, t =one-fourth T_{H}, t=one-half T_{H}, t=three-fourths T_{H}, and t=T_{H}. As the hold time progresses from t=0 to t=T_{H}, first portion 506 becomes smaller as second portion 508 becomes larger. Visualization device 116 may update, e.g., change the sizes of, first portion 506 and second portion 508 at a relatively slow rate, such that the wearer of visualization device 516 sees the updates as discrete events or may update may update first portion 506 and second portion 508 at a rate sufficiently fast that first portion 506 appears to be continuously shrinking as second portion 508 continuously expands.

FIG. 5C shows an alternate implementation of visual cue 500. In the example of FIG. 5C, visual cue completely surrounds ROI 502 in a circular manner. Similar to FIGS. 5A and 5B, in FIG. 5C, a color changes around ROI 502 as the holding period progresses. The concepts described with respect to FIGS. 5B and 5C may also be implemented with other shapes.

FIG. 5D shows another alternate implementation of visual cue 500. In contrast to FIGS. 5A-5C, where second portion 508 expands around ROI 502 as the hold time progresses, in FIG. 5D, second portion 508 expands towards ROI 502 as the hold time progresses. The concepts described with respect to FIG. 5D may also be implemented with other shapes. The visual cues of FIGS. 5B-5D may be conceptualized as, and potentially implemented as, background objects (e.g., the first portions) that are covered by expanding foreground objects (e.g., the second portions). In some implementations, the foreground object may expand without the presence of a background object. For example, the visual cue may include second portion 508 without first portion 506.

FIG. 5E shows another alternate implementation of visual cue 500. In contrast to FIGS. 5A-5D where first portion 506 and second portion 508 change in size as the hold time progresses, in FIG. 5E, visual cue 500 changes color as the hold time progresses. Visual cue 500 may, for example, transition from red to yellow to green as the hold time of progresses or transition from a light shade of blue to a darker blue as the hold time progresses. The concepts described with respect to FIG. 5D may also be implemented with other shapes and colors.

It should be understood that FIGS. 5A-5E represent merely a few of the many forms which visual cue 500 may take. Visual cue 500 may essentially be any shape or color, and in some implementations, may be semi-transparent so as to not fully obstruct a user's view of anatomy or instruments. In some implementations, surgical assistance system 100 may position visual cue 500, e.g., position the opening of visual cue 500 in FIG. 5A or 5B, at a location of interest, such as an instrument or portion of anatomy, so as to further reduce any obstruction caused by visual cue 500. For visual cue 500 in FIGS. 5A-5E, surgical assistance system 100 may update visual cue 500 to reflect the progress of the registration based, for example, on a percentage of points within region of interest 502 that have been acquired.

Referring back to FIGS. 5B-5E, t=0 generally corresponds to zero points having been acquired; t=one-fourth T_{H} corresponds to approximately 25% of points having been acquired; t=one-half T_{H} corresponds to approximately 50% of points having been acquired; t=three-fourths T_{H} corresponds to approximately 75% of points having been acquired; and t=T_{H} corresponds to 100% of points having been acquired.

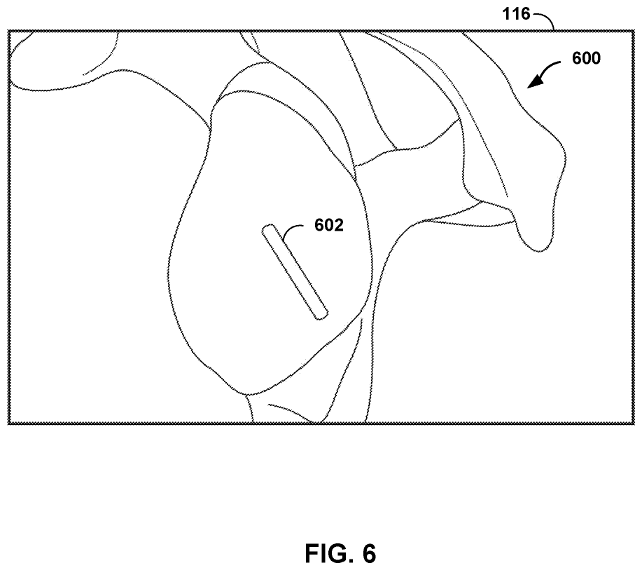

FIG. 6 is a conceptual diagram of virtual guidance that may be provided by surgical assistance system 100 after registration is complete. As shown in FIG. 6, a surgeon may view a portion of a scapula 600 through visualization device 116 with a gaze line substantially parallel (e.g., closer to parallel than perpendicular) to an axis of the surgical step being performed. For instance, as shown in FIG. 6 where visualization device 116 displays virtual axis 602 to guide use of a rotating tool to perform a surgical step on scapula 600, the axis of the surgical step being performed may correspond to virtual axis 602. As such, the surgeon may view scapula 600 through visualization device 116 with a gaze line substantially parallel to an axis of virtual axis 602.

As discussed above, in some examples, the surgeon may utilize one or more tools to perform work on portion of a patient's anatomy (e.g., scapula 600, etc.). For instance, the surgeon may utilize a driver to drive (e.g., provide rotational power to) a rotating tool. Examples of rotating tools include, but are not limited to, guide pins (e.g., self-tapping guide pins, such as guide 680), reaming tools, drill bits, and screw drivers.

As also discussed above, surgical assistance system 100 may provide virtual guidance to assist the surgeon in performing surgical steps. For instance, as shown in FIG. 6, visualization device 116 of surgical assistance system 100 may display virtual axis 602 (e.g., a virtual planned insertion axis object) to guide use of a rotating tool to perform a surgical step on scapula 600. The surgeon may achieve correct performance of the surgical step by aligning a shaft of the rotating tool with virtual axis 602, activating a driver of the rotating tool, and advancing the shaft of the rotating tool along the displayed virtual axis.

In some examples, surgical assistance system 100 may track a position and/or orientation of the rotating tool and used the position and/orientation to display the virtual guidance. For instance, surgical assistance system 100 may track the position and/or orientation of the rotating tool based on data generated by one or more depth cameras and/or optical cameras (e.g., depth cameras and/or optical cameras of visualization device 116 or one or more other devices). However, in some scenarios, the rotating tool, the driver of the rotating tool, and/or various tools used by the surgeon may obscure or otherwise interfere with the ability of one or more sensors of visualization device 116 to directly track the position of the rotating tool (e.g., guide 680 or any other insertable item). For instance, the driver may obstruct a line of sight between the depth/optical cameras of visualization device 116 and the rotating tool. Without such a line of sight, it may not be possible for surgical assistance system 100 to directly track the position and/or orientation of the rotating tool or directly tracking the position and/or orientation of the rotation tool in the presence of such obstruction may be insufficiently reliable.

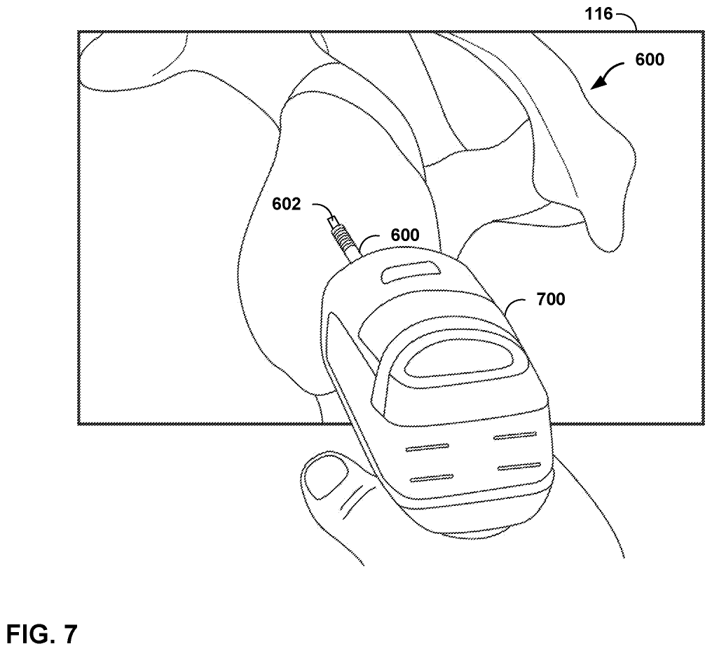

FIG. 7 is a conceptual diagram of tools obscuring a portion of virtual guidance provided by an MR system. As can be seen in FIG. 7, when viewing with a gaze line substantially parallel to the axis of the surgical step being performed (i.e., substantially parallel to virtual axis 602) driver 7800 at least partially obscures the line of sight between the depth/optical cameras of visualization device 116 and guide 680 displayed virtual guidance (i.e., virtual axis 602). With the line of sight obscured, it may be difficult for surgical assistance system 100 to be able to track the position and/or orientation of the rotating tool based on data generated by one or more depth cameras and/or optical cameras of visualization device 116.

Surgical assistance system 100 may track a position and/or orientation of a physical tracking tool that attaches to a tool in order to determine a position and/or orientation of the tool. Where the tool is a rotating tool, the physical tracking tool may include a channel through which the shaft of the rotating tool is inserted, and one or more physical tracking features that each include a plurality of planar faces. In operation, surgical assistance system 100 may utilize data generated by the depth/optical cameras of visualization device 116 to determine a position and/or orientation of at least one face of the plurality of planar faces. Surgical assistance system 100 may then adjust the determined position/orientation by a pre-determined offset that represents a displacement between the at least one face and the channel. The adjusted position/orientation represents the position/orientation of the tool in the channel. In this way, surgical assistance system 100 may determine the position/orientation of a tool where a line of sight between sensors of surgical assistance system 100 and the tool (e.g., guide 680) is at least partially obstructed. Further details of some examples of a physical tracking tool are discussed below with reference to FIGS. 8A-8E.

Surgical assistance system 100 may display the virtual guidance using the determined position/orientation of the tool. For instance, using the determined position/orientation of guide 680, surgical assistance system 100 may display virtual guidance as described below.

FIGS. 8A-8C illustrate various views of one example of a physical tracking tool, in accordance with one or more techniques of this disclosure. As shown in FIGS. 8A-8C, physical tracking tool 800 may include a main body 802, a physical tracking feature 808, and a handle 812.

Main body 802 may define a channel configured to receive a tool. For instance, main body 802 may define a channel 801 that is configured to receive a shaft of a rotating tool, such as guide 680 of FIG. 7. Channel 801 may have a primary axis that controls movement of a received tool, such as an item insertable into a bone of a patient. For instance, where channel 801 is configured to receive a shaft of an insertable item, channel 801 may have a longitudinal axis (e.g., longitudinal axis 816) about-which the shaft may rotate. Channel 801 may be configured to receive the insertable item in the sense that channel 801 is sized such that an inner dimension of channel 801 is slightly larger than an outer dimension of the insertable item. For instance, where the tool is a rotating tool, channel 801 may be cylindrical and have an inner diameter that is slightly larger than an outer diameter of a shaft of the insertable item. In this way, the shaft of the insertable item may spin within channel 801 but may be confined to rotation about longitudinal axis 816.

Channel 801 may extend all the way through main body 802 such that channel 801 is open at both distal end 814 and proximal end 806. Therefore, an insertable item may be inserted into proximal end 806, advanced through channel 801, and come out of distal end 814.

Physical tracking tool 800 may include one or more physical tracking features. The one or more physical tracking features may be attached to main body 802, handle 812, or another other component of physical tracking tool 800. In the example of FIGS. 8A-8C, the one or more physical tracking features include physical tracking feature 808. In some examples, each of the physical tracking features is a polyhedron that includes a plurality of planar faces. For instance, as shown in the example of FIGS. 8A-8C, physical tracking feature 808 may be a cube that includes a top face 810 (i.e., face that is farthest away from main body 802) and a plurality of side faces 818A-918D (collectively, “side faces 818”). In examples where physical tracking feature 808 is a cube, side faces 818 may include exactly four side faces in addition to top face 810. Other example polyhedrons that physical tracking features may be shaped as include, but are not limited to, pyramids (e.g., tetrahedron), octahedrons, prisms, and the like.

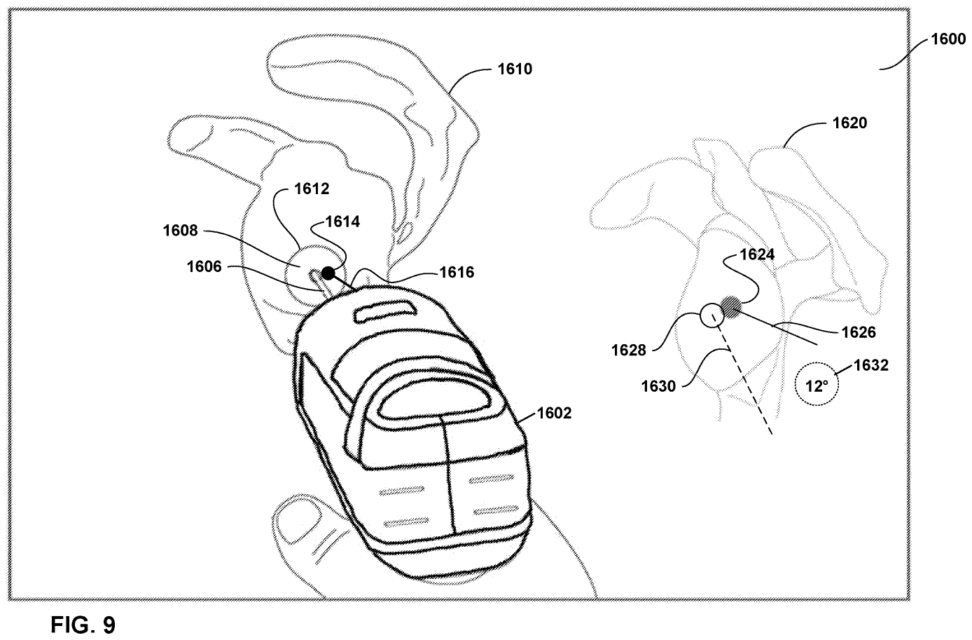

The faces of the one or more physical tracking features may each include a respective graphical pattern. The graphical patterns may have visual characteristics that renders them highly detectable to electronic sensors. For instance, as shown in FIGS. 8A-8C, top face 810 and side faces 818 may each include graphical patterns that include high contrast features (i.e., white shapes on a black background). In some examples, each of the graphical patterns may include a rectangular perimeter. For instance, each of top face 810 and side faces 818 may include a solid rectangular background (e.g., a black square in FIGS. 8A-9C) having four corners (e.g., side face 818A is illustrated as having corners 830A-830D (collectively, “corners 830”) in FIG. 8B). Surgical assistance system 100 may be configured to determine a relative location of a particular face (e.g., relative to channel 801) based on the graphical pattern included on the particular face. In other words, surgical assistance system 100 may segment image data generated by one or more cameras of visualization device 116 into regions by using the perimeters of the solid rectangular backgrounds. Within each region, surgical assistance system 100 may compare the patterns with a database of known graphical patterns to identify which pattern is included in the region. Surgical assistance system 100 may utilize the database to determine which face corresponds to the identified pattern. For instance, surgical assistance system 100 may determine that top face 810 corresponds to a first pattern, side face 818A corresponds to a second pattern, side face 818B corresponds to a third pattern, side face 818C corresponds to a fourth pattern, and side face 818D corresponds to a fifth pattern. Surgical assistance system 100 may determine a respective offset from each identified face to another portion of physical tracking tool 800 (e.g., channel 801).

Surgical assistance system 100 may further determine, within each identified region, coordinates (e.g., three-dimensional coordinates) of one or more corners of the identified region. For instance, surgical assistance system 100 may determine, based on image data generated by an RGB camera of visualization device 116, two-dimensional coordinates of corners 830 of side face 818A. Based on intrinsic properties of the RGB camera and a pre-determined size of the solid rectangular background of side face 818A, surgical assistance system 100 may convert the two-dimensional coordinates of corners 830 into three-dimensional coordinates (e.g., using the Perspective-n-Point algorithm). Surgical assistance system 100 may adjust the determined coordinates by the determined offset to determine a position and/or orientation of a portion of physical tracking tool (e.g., channel 801). As discussed above, when the insertable object is placed in channel 801, the position and/or orientation of the insertable object roughly corresponds to the position and/or orientation of channel 801. As such, by determining the position and/or orientation of channel 801, surgical assistance system 100 effectively determines the position and/or orientation of the insertable object. In this way, surgical assistance system 100 may track a position and/or orientation of an insertable object using physical tracking tool 800.

In some examples, at least one face of the plurality of side faces may be orthogonal to longitudinal axis 816. For instance, as shown in FIGS. 8A-8C, side face 818A and side face 818C may be orthogonal to longitudinal axis 816. However, for some uses of physical tracking tool 800, the surgeon's gaze (and thus the “gaze” of sensors of visualization device 116) may not be directly along longitudinal axis 816. Detection of physical tracking tool 800 may be improved when the “gaze” of sensors of visualization device 116 is more orthogonal to a face of physical tracking feature 808.

In some examples, physical tracking feature 808 may be rotated such that at least one of the planar faces is not orthogonal to longitudinal axis 816. For instance, an angle between at least one side face of side faces 818 and a plane orthogonal to longitudinal axis 816 (i.e., angle 850 of FIG. 8D) may be greater than or equal to 10 degrees. As one specific example, the angle between side face 818A and a plane orthogonal to longitudinal axis 816 (i.e., angle 850) may be approximately 20 degrees (e.g., +/−10%). By rotating physical tracking feature 808 may be rotated such that at least one of the planar faces is not orthogonal to longitudinal axis 816, the probability that multiple faces of physical tracking feature 808 will be within the line of sight of visualization device 116 may be increased. As the number of faces of physical tracking feature 808 within the line of sight of visualization device 116 increases, the number of points identified by surgical assistance system 100 increases. An increase in the number of points identified may lead to an increase in the accuracy of the tracking of physical tracking feature 808.

In some examples, physical tracking tool 800 may be ambidextrous such that physical tracking tool 800 can be similarly operated by right-handed and left-handed surgeons. In other examples, physical tracking tool 800 may be or may not be ambidextrous (i.e., physical tracking tool 800 may come in right-handed or left-handed configurations). Where physical tracking tool 800 is not ambidextrous, handle 812 of physical tracking tool 800 may be configured such that, when handle 812 is gripped by a non-dominant hand of the surgeon, physical tracking feature 808 may be “on top” (i.e., located above channel 801). In the example of FIGS. 8A-8C where handle 812 is located on the left when tracking feature 808 is “on top”, handle 812 may be considered to be configured to be gripped by a left hand.

As mentioned above, in some examples, physical tracking tool 800 may be ambidextrous. In some examples, to make physical tracking tool 800 ambidextrous, physical tracking tool 800 may include a plurality of physical tracking features 808 disposed at different positions around main body 802. For instance, as shown in FIG. 8E, physical tracking tool 800A may include physical tracking features 808A and 808B disposed on opposite sides of main body 802. As such, regardless of which hand physical tracking tool 800A is held in, one of physical tracking features 808A and 808B will be on top, and thus be in the line of sight of sensors of visualization device 116. Where a physical tracking tool includes a plurality of physical tracking features, corresponding faces of the respective physical tracking features may have different graphical patterns. For instance, side face 818A1 and side face 818A2 may be considered to be corresponding faces and may have identical graphical patterns or may have different graphical patterns. Additionally, it is noted physical tracking features 808A and 808B may be rotated (e.g., similar to as shown in FIG. 8D).

Handle 812, in some examples, may be integral to main body 802. For instance, handle 812 may be permanently attached to main body 802 and/or may be formed as a single component with main body 802. In other examples, handle 812 may be removable from main body 802. For instance, handle 812 may be removable and “flappable” such that placement of handle 812 one way or the other renders physical tracking tool 800 in a right-handed or left-handed configuration.

A distance between the one or more physical tracking features and main body 802 may be varied. In some examples, physical tracking feature 808 may be positioned relatively closed to main body 802. For instance, a distance between top face 810 and longitudinal axis 816 may be less than 50 millimeters (mm) or less than 30 mm. In some examples, physical tracking feature 808 may be positioned relatively far from to main body 802. For instance, a distance between top face 810 and longitudinal axis 816 may be greater than 50 mm or greater than 70 mm. Where the one or more physical tracking features include a plurality of physical tracking features, the distances between the tracking features and the main body may be the same for all tracking features or may be different.

While described above as being incorporated in a physical tracking tool configured to guide an insertable object, the techniques of this disclosure are not so limited. For instance, the physical tracking features described herein (e.g., with reference to FIGS. 8A-8F and 10) may be attached to other tools (e.g., directly incorporated into the tools, or attached via some other means, such as clips) in order to facilitate tracking of said tools. As one specific example, one or more physical tracking features (e.g., similar to physical tracking feature 808) may be attached to a reamer and surgical assistance system 100 may utilize said physical tracking features to track a depth of reaming.

Surgical assistance system 100 may obtain sensor data generated by one or more sensors of visualization device 116. For instance, one or more processors 214 may obtain image data from one or more optical camera(s) 230 (or other optical sensors) and/or one or more depth camera(s) 232 (or other depth sensors) of visualization device 116. The image data may depict a scene including a physical tracking tool comprising one or more physical tracking features that each comprise a plurality of planar faces, each planar face of the plurality of planar faces including different a graphical pattern of a plurality of pre-determined graphical patterns (e.g., physical tracking tool 800 of FIG. 8A).

Surgical assistance system 100 may determine, based on the sensor data, coordinates of a plurality of points on graphical patterns on the tracking tool. For instance, one or more processors 214 may process the image data to identify coordinates of one or more corners of a rectangular perimeter of a particular graphical pattern (e.g., identify two-dimensional coordinates of corners 830 of the graphical pattern on side face 818A, and then convert said two-dimensional coordinates into three-dimensional coordinates (e.g., using Perspective-n-Point)). One or more processors 214 may identify, based on the sensor data, a graphical pattern of the plurality of pre-determined graphical patterns that corresponds to the particular graphical pattern. For instance, one or more processors 214 may determine that the graphical pattern depicted in the image data is the graphical pattern illustrated on side face 818A in FIG. 8A. One or more processors 214 may obtain a physical offset between a planar face corresponding to the identified graphical pattern. For instance, responsive to determining that the image data includes the graphical pattern illustrated on side face 818A in FIG. 8A, one or more processors 214 may obtain (e.g., from memory 216) values that represent a physical displacement between corners of the graphical pattern illustrated on side face 818A in FIG. 8A and channel 801. One or more processors 214 may obtain the offsets for each graphical pattern detected in the image data or a subset of the graphical patterns detected in the image datax

Surgical assistance system 100 may determine, based on the determined coordinates, a position and/or an orientation of an insertable object guided by the physical tracking tool. For instance, one or more processors 214 may adjust the determined coordinates of a plurality of points on the graphical patterns based on the physical offset. As one example, where one or more processors 214 determines the coordinates of corners of a rectangular perimeter as (x₁,y₁,z₁; x₂,y₂,z₂; x₃,y₃,z₃; and x₄,y₄ ,z₄) and determines the physical offset as (x_{offset}, y_{offset}, z_{offset}), one or more processors 214 may average the determined coordinates to determine coordinates of a centroid of the graphical pattern (e.g., x_{avg}, y_{avg}, z_{avg}) and add the coordinates of the centroid with the physical offset to determine a coordinate of channel 801. One or more processors 214 may repeat this process for each, or a subset, of the graphical patterns detected in the image data.

FIG. 9 is a conceptual diagram illustrating a fourth example MR scene in accordance with an example of this disclosure. As discussed above, the surgeon may utilize one or more tools to insert an insertable item into a bone (e.g., scapula, humerus, etc.) of a patient. In the example of FIG. 9, the surgeon may use a drill, such as a drill 1602, to insert an insertable item, such as a drill bit, pin, screw, or other type of insertable item into a bone of the patient. Although this disclosure describes FIG. 9 with respect to drill 1602, other types of surgical tools (e.g., an impactor) may be used to insert an insertable item into the bone of the patient. For instance, in another example, the surgeon may use an impactor to tap an insertable item, such as a surgical nail or awl, into a bone of the patient.

One of the issues confronting surgeons when using surgical tools to insert insertable items into bones of patients is that the surgical tools may fully or partially block the surgeons' look down the axes of the insertable items. This inability to look down the axes of an insertable item may hinder the ability of a surgeon to insert the insertable item into the bone of the patient at the correct angle. The examples described with respect to FIG. 9 and FIG. 10 may address such issues. That is, in the example of FIG. 9 and FIG. 10, surgical assistance system 100 to generate MR scene 1600 that includes information to help the surgeon insert an insertable item 1606 in a real-world bone 1608 at the correct angle.

As shown in the example of FIG. 9, a surgeon is using drill 1602 to insert an insertable item 1604 into real-world bone 1608. Furthermore, in the example of FIG. 9, surgical assistance system 100 superimposes a first virtual bone model 1610 over real-world bone 1608 in MR scene 1600. Virtual bone model 1610 is a 3D virtual model of real-world bone 1608 or a portion of real-world bone 1608. For instance, in examples such as the example of FIG. 9 where real-world bone 1608 is a patient's scapula, virtual bone model 1610 is a virtual model of the patient's scapula. Virtual bone model 1610 may be based on medical imaging of the real-world bone 1608.

Surgical assistance system 100 registers virtual bone model 1610 with real-world bone 1608 and maintains an alignment in MR scene 1600 between virtual bone model 1610 and real-world bone 1608. Thus, as the surgeon's viewing position of real-world bone 1608 changes, surgical assistance system 100 may scale and/or reorient virtual bone model 1610 within MR scene 1600 so that virtual bone model 1610 appears to remain aligned with real-world bone 1608. In this way, virtual bone model 1610 may help the surgeon understand the orientation of real-world bone 1608 within the body of the patient despite portions of real-world bone 1608 being obscured by other tissues (e.g., muscle, skin, etc.) of the patient.

Furthermore, in the example of FIG. 9, surgical assistance system 100 may define a window 1612 in virtual bone model 1610. Window 1612 may be an opening through virtual bone model 1610 through which the surgeon is able to see a planned insertion point of insertable item 1606 in real-world bone 1608. Thus, in the example of FIG. 9, while surgical assistance system 100 may superimpose virtual bone model 1610 over parts of real-world bone 1608, window 1612 allows the surgeon to see the portion of real-world bone 1608 on which the surgeon is working.

Surgical assistance system 100 may also include a virtual target insertion point object 1614 in MR scene 1600. Surgical assistance system 100 positions virtual target insertion point object 1614 in MR scene 1600 at a position corresponding to a location on real-world bone 1608 where the surgeon is to insert insertable item 1606 into real-world bone 1608 according to a surgical plan for the surgical procedure. In some examples, virtual target insertion point object 1614 is not a separate virtual object but forms part of the same virtual object as virtual bone model 1610.

In addition, surgical assistance system 100 may include a virtual target axis object 1616 in MR scene 1600. Virtual target axis object 1616 corresponds to a planned insertion axis along which the surgeon is to insert insertable item 1606 into bone 1608. Thus, virtual target axis object 1616 may appear to extend outwardly from bone 1608 and virtual bone model 1610 at an angle relative to the surface of bone 1608 and virtual bone model 1610 at which insertable item 1606 is to be inserted into bone 1608. In some examples, virtual target insertion point object 1614, virtual target axis object 1616, and virtual bone model 1610 may be separate virtual objects or two or more of virtual target insertion point object 1614, virtual target axis object 1616 may be the same virtual object.

As shown in the example of FIG. 9, it may be difficult at times for the surgeon to see one or more of virtual insertion point object 1614 or virtual target axis object 1616, or portions thereof, because virtual insertion point object 1614 and/or virtual target axis object 1616, or portions thereof, are obscured by drill 1602 (or another surgical tool), the surgeon's own hand, or other objects. For instance, in the example of FIG. 9, only a small portion of virtual target axis object 1616 is visible because a remainder of virtual target axis object 1616 is obscured by drill 1602. Not being able to see virtual insertion point object 1614 and/or virtual target axis object 1616, or portions thereof, may hinder the ability of the surgeon to insert insertable item 1606 into bone 1608 at a planned insertion point and/or insert insertable item 1606 into bone 1608 at a planned insertion angle. This may lead to frustration on the part of the surgeon and/or slow surgical times.

In accordance with one or more techniques of this disclosure, surgical assistance system 100 may include a second virtual bone model 1620. Virtual bone model 1620 may include a 3D virtual model of real-world bone 1608 or a portion of real-world bone 1608 into which the surgeon plans to insert insertable item 1606. In some examples, virtual bone model 1620 may be patient-specific. In other examples, virtual bone model 1620 may be generic across multiple patients. Surgical assistance system 100 may position virtual bone model 1620 in MR scene 1600 at a position far enough away from bone 1608 and drill 1602 (or other surgical tool) that the surgeon may be able to see both virtual bone model 1610 and virtual bone model 1620 simultaneously and without virtual bone model 1610 being obscured by drill 1602 (or other surgical tool) or another object, such as the surgeon's hand.

Surgical assistance system 100 may align virtual bone model 1610 in 3D space in the same orientation as the bone 1608. Surgical assistance system 100 may use a registration process, such as any of the registration processes described elsewhere in this disclosure, to determine how to align virtual bone model 1620 in 3D space with bone 1608. Thus, as the surgeon moves around to view bone 1608 from different angles, surgical assistance system 100 reorients virtual bone model 1620 in 3D space so that the surgeon is able to see virtual bone model 1620 from the same angle as bone 1608.

Additionally, in the example of FIG. 9, surgical assistance system 100 may include a virtual target insertion point object 1624 in MR scene 1600. Surgical assistance system 100 positions virtual target insertion point object 1624 on virtual bone model 1620 that corresponds to the location on real-world bone 1608 where the surgeon is to insert insertable item 1606 into real-world bone 1608 according to a surgical plan for the surgical procedure. In some examples, virtual target insertion point object 1614 is not a separate virtual object but forms part of the same virtual object as virtual bone model 1610.

Surgical assistance system 100 may also include a virtual planned insertion axis 1622 in MR scene 1600. Virtual target axis object 1626 corresponds to a planned insertion axis along which the surgeon is to insert insertable item 1606 into bone 1608. Thus, virtual target axis object 1618 may appear to extend outwardly from virtual bone model 1614 at an angle relative to the surface of virtual bone model 1614 at which insertable item 1606 is to be inserted into bone 1608.

Furthermore, in the example of FIG. 9, surgical assistance system 100 may include a virtual current insertion point object 1268 in MR scene 1600. Virtual current insertion point object 1268 is a virtual object that corresponds to a location on virtual bone model 1620 where insertable item 1606 is currently in contact with bone 1608 or where insertable item 1606 would enter bone 1608 if insertable item 1606 were translated along a lengthwise axis of insertable item 1606. Surgical assistance system 100 may update the position of virtual current insertion point object 1268 to maintain this correspondence as the surgeon moves insertable item 1606. In this way, virtual current insertion point object 1268 may help the surgeon understand a position of insertable item 1606 relative to bone 1608. By comparing the locations of virtual target insertion point object 1614 and virtual current insertion point object 1268, the surgeon may be able to determine whether a tip of insertable item 1606 is at a planned insertion location on bone 1608.

In the example of FIG. 9, surgical assistance system 100 also includes a virtual current axis object 1630. Virtual current axis object 1630 is a virtual object having a lengthwise axis having a spatial orientation relative to virtual bone model 1620 that corresponds to a spatial orientation of the lengthwise axis of insertable item 1606 relative to bone 1608. Surgical assistance system 100 may update the position of virtual current axis object 1230 to maintain this correspondence as the surgeon moves insertable item 1606. Thus, if the surgeon tilts insertable item 1606 in a superior direction relative to bone 1608 by 5°, surgical assistance system 100 updates virtual bone model 1620 within MR scene 1600 such that virtual current axis object 1630 appears to move in the superior direction relative to virtual bone model 1620 by 5°. By comparing the orientations of virtual target axis object 1626 and virtual current axis object 1630, the surgeon may be able to determine whether the lengthwise axis of insertable item 1606 is aligned with the planned insertion axis along which the surgeon is to insert insertable item 1606 into bone 1608.

In some instances, depending on the position of the surgeon, the lengthwise axis of insertable item 1606 may be aligned with a gaze line of the surgeon. When this occurs, the surgeon may be unable to see bone 1608 because bone 1608 would be blocked by drill 1602. When this occurs, virtual bone model 1620 is also shown from a perspective down the lengthwise axis of insertable item 1606. Thus, using virtual bone model 1620, the surgeon may be able to have a view that may be equivalent to a view of bone 1608 that would otherwise be blocked by drill 1602.

Furthermore, in the example of FIG. 9, MR scene 1600 includes an angle indicator 1632. Angle indicator 1632 indicates a 3D angle between the planned insertion axis and the lengthwise axis of insertable item 1606.

In some examples, virtual target insertion point object 1624, virtual target axis object 1626, and virtual bone model 1620 may be separate virtual objects or two or more of virtual target insertion point object 1624, virtual target axis object 1626, or virtual bone model 1620 may be the same virtual object. In some examples, virtual current insertion point object 1628 and virtual current axis object 1630 may be separate virtual objects or virtual current insertion point object 1628 and virtual current axis object 1630 may be the same virtual object.

FIG. 10 is a flowchart illustrating an example operation 1000 of the surgical system corresponding to the MR scene of FIG. 9. However, the operation 1000 of FIG. 10 is not so limited. Like the other flowcharts of this disclosure, FIG. 10 is provided as an example. Other examples may include more, fewer, or different actions.

As shown in the example of FIG. 10, surgical assistance system 100 obtains virtual bone model 1620 (FIG. 9) (1002). Virtual bone model 1620 may be a 3D virtual model of real-world bone 1608. Virtual bone model 1620 may be located at positions in a virtual coordinate system.

In some examples, surgical assistance system 100 also obtains virtual target axis object 1626. Virtual target axis object 1626 may be located in the virtual coordinate system such that virtual target axis object 1626 is oriented relative to virtual bone model 1620 such that a lengthwise axis of virtual target axis object 1626 intersects virtual bone model 1620 at a planned insertion point of insertable item 1606 along a planned insertion axis for insertable item 1606.

Surgical assistance system 100 may obtain virtual bone model 1620, virtual target insertion point object 1624, and virtual target axis object 1626 from previously stored data representing a surgical plan for the surgical procedure. In examples that include virtual bone model 1610, virtual target insertion point object 1614, and/or virtual target insertion axis object 1616, surgical assistance system 100 may also retrieve these virtual objects from previously stored data representing the surgical plan for the surgical procedure. In some examples, surgical assistance system 100 may obtain one or more of the virtual objects (e.g., virtual bone model 1610, virtual target insertion point object 1614, virtual target insertion axis object 1616, virtual bone model 1620, virtual target insertion point object 1624, and virtual target axis object 1626) by generating one or more aspects of the virtual objects during the surgical procedure in response to indications of user input from the surgeon and/or other users. For instance, the surgeon may provide user input to adjust the planned insertion axis during the surgical procedure.

Surgical assistance system 100 may store positions of virtual objects (e.g., virtual bone model 1610, virtual target insertion point object 1614, virtual target insertion axis object 1616, virtual bone model 1620, virtual target insertion point object 1624, virtual target axis object 1626, etc.) in a virtual coordinate system. The virtual coordinate system is a coordinate system for tracking positions of virtual objects.

Surgical assistance system 100 may determine locations of insertable item 1606 and real-world bone 1608 in a real-world coordinate system (1004). In some examples, to determine the location in the real-world coordinate system of insertable item 1606 and to continue tracking the location of insertable item 1606 in the real-world coordinate system, surgical assistance system 100 may determine coordinates of two or more landmarks on insertable item 1606 in a real-world coordinate system. The two or more landmarks may be different points on the lengthwise axis of insertable item 1606. In the context of a drill bit, screw, or pin, the lengthwise axis of insertable item 1606 may be an axis about which insertable item 1606 rotates. In some examples, surgical assistance system 100 may determine the 3D orientation of insertable item 1606 based on a 3D orientation of a physical tracking feature of a physical tracking tool that includes a body defining a channel through which insertable item 1606 passes during insertion of insertable item 1606 into bone 1608, as described in detail elsewhere in this disclosure.

Additionally, surgical assistance system 100 may perform a registration process to register the real-world coordinate system with the virtual coordinate system (1006). Registering the real-world coordinate system with the virtual coordinate system may include determining a transform function between the real-world coordinate system and the virtual coordinate system. In some examples, as part of the registration process, surgical assistance system 100 may include virtual bone model 1610 in MR scene 1600 and receive one or more indications of user input to move virtual bone model 1610 to a location overlapping real-world bone 1608. In such examples, surgical assistance system 100 may then identify corresponding landmarks on virtual bone model 1610 and real-world bone 1608. Surgical assistance system 100 may then determine a transform function for mapping between the virtual coordinates of the landmarks of virtual bone model 1610 and real-world coordinates of the landmarks of real-world bone 1608.

As described above, registering the real-world coordinate system with the virtual coordinate system may require a hold time, where a wearer of visualization device 116 needs to maintain a relatively steady gaze at a specific point or region of interest, such as a region that includes the two or more landmarks. It may also be desirable for the wearer of visualization device 116 to hold physical tracking tool 800 relatively steady and in a constant position during the hold time. To aid the wearer of visualization device 116 in maintaining a steady gaze and holding physical tracking tool 800 relatively steady, visualization device 116 may be configured to output, via UI 222, a visual cue. As described with respect to FIGS. 5A-5E, the visual cue may fully or partially surround the region of interest.

Furthermore, in the example of FIG. 10, surgical assistance system 100 may include, in MR scene 1600, virtual bone model 1620, virtual target axis object 1626, and virtual current axis object 1630 at a location removed from real-world bone 1608 (1008). In other words, virtual bone model 1620, virtual target axis object 1626, and virtual current axis object 1630 are not superimposed on real-world bone 1608. For instance, in the example of FIG. 10, surgical assistance system 100 may include virtual bone model 1620, virtual target axis object 1626, and virtual current axis object 1630 at a location to the right of real-world bone 1608. In some examples, surgical assistance system 100 may also include virtual target insertion point object 1624 and virtual current insertion point object 1628 in MR scene 1600.

Additionally, in the example of FIG. 10, surgical assistance system 100 may include virtual bone model 1610 in MR scene 1600 at a location superimposed on real-world bone 1608 (1010). In some examples, surgical assistance system 100 may also include one or more of virtual target insertion point object 1614 or virtual target axis object 1616 in MR scene 1600.

In the example of FIG. 10, surgical assistance system 100 may determine whether the lengthwise axis of insertable item 1606 is aligned with a planned insertion axis (1012). When the lengthwise axis of insertable item 1606 is aligned with the planned insertion axis, virtual current insertion axis object 1630 is aligned with virtual target insertion axis object 1626. Furthermore, when the lengthwise axis of insertable item 1606 is aligned with the planned insertion axis, the lengthwise axis of insertable item 1606 is aligned with virtual target insertion axis 1616.

In some examples, surgical assistance system 100 may determine that the lengthwise axis of insertable item 1606 is aligned with the planned insertion axis if a difference between a current insertion point of insertable item 1606 and a planned insertion point of insertable item 1606 is less than a first threshold and a difference between an angle of insertable item 1606 relative to a surface of bone 1608 and an angle of the planned insertion axis relative to bone 1608 is less than a second threshold. In this example, the first and second thresholds may be defined in a way that distances and angles less than the first and second thresholds are acceptable for successful performance of the surgical procedure.

In response to determining that the lengthwise axis of insertable item 1606 is aligned with planned insertion axis (“YES” branch of 1012), surgical assistance system 100 may generate user feedback indicating alignment of the lengthwise axis of insertable item 1606 with planned insertion axis (1014). For example, surgical assistance system 100 may include, in MR scene 1600, a virtual object that appears like a glow or halo around virtual target axis object 1630 when the lengthwise axis of insertable item 1606 is aligned with the planned insertion axis. In some examples, surgical assistance system 100 may generate an audible indication that the lengthwise axis of insertable item 1606 is aligned with the planned insertion axis. Although not shown in the example of FIG. 10, surgical assistance system 100 may generate user feedback when the lengthwise axis of insertable item 1606 is not aligned with the planned insertion axis. For example, surgical assistance system 100 may generate audible indications that the lengthwise axis of insertable item 1606 is not aligned with the planned insertion axis. In some examples, surgical assistance system 100 may change a color of one or more of virtual target axis object 1616, virtual target axis object 1626, or virtual current axis object 1630 based on how far the lengthwise axis of insertable item 1606 is from being aligned with the planned insertion axis.

Regardless of whether the lengthwise axis of insertable item 1606 is aligned with the planned insertion axis, surgical assistance system 100 may determine an updated real-world position of insertable item 1606 (1016). For example, as described elsewhere in this disclosure, surgical assistance system 100 may track the position of a physical tracking feature of a physical tracking tool. In this example, surgical assistance system 100 may determine the updated position of insertable item 1606 based on an updated position of the physical tracking feature.

Surgical assistance system 100 may update the orientation of the virtual current axis object 1630 based on the updated orientation of insertable item 1606 so that the orientation of virtual current axis object 1630 relative to virtual bone model 1620 corresponds to the updated orientation of the lengthwise axis of insertable item 1606 relative to bone 1608 (1018). Surgical assistance system 100 may continue performing actions 1016 and 1018 multiple times (e.g., until a step of the surgical procedure that involves inserting insertable item 1606 into bone 1608 is complete).

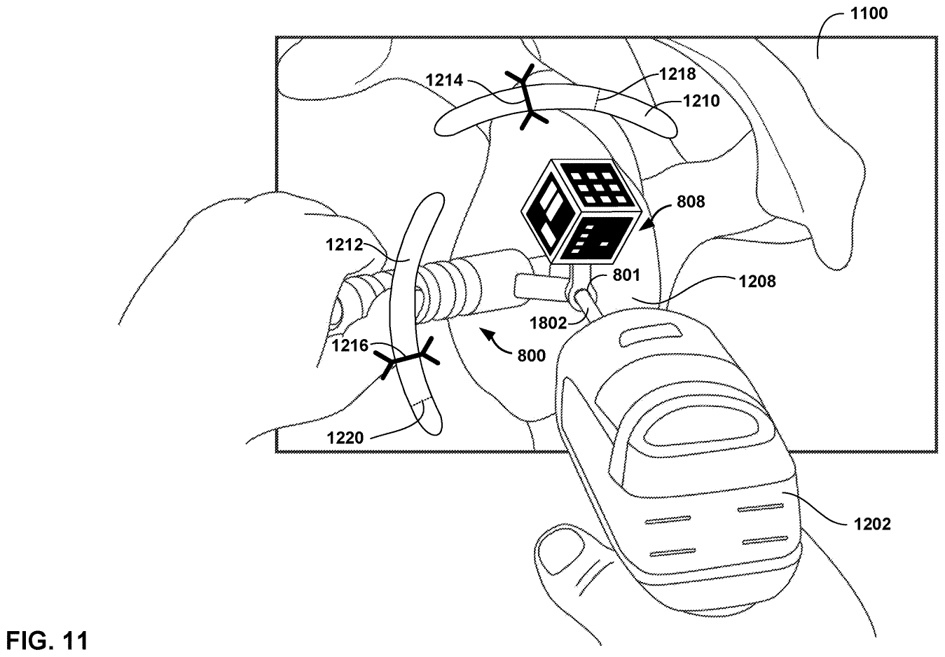

FIG. 11 is a conceptual diagram illustrating an MR scene 1100 in accordance with an example of this disclosure. As shown in MR scene 1100 the surgeon may insert insertable item 1802 into channel 801 of physical tracking tool 800. Surgical assistance system 100 may track a position and/or orientation of insertable item 1802 using any of the techniques described herein. For example, surgical assistance system 100 may track the position and/or orientation of insertable item 1802 using the techniques discussed above with reference to FIGS. 8A-8F. Surgical assistance system 100 may utilize the determined position and/or orientation of insertable item 1802 to display virtual guidance.

Drill 1202 may be an example of a driver, such as driver 700 (FIG. 7). Thus, in the example of FIG. 11, surgical assistance system 100 may generate MR scene 1100 that includes information to help the surgeon insert an insertable item 1802 in a real-world bone 1208 at the correct angle. In the example of FIG. 11, real-world bone 1208 is a scapula. In other examples, real-world bone 1208 may be another type of bone or set of bones. Furthermore, in the example of FIG. 11, the surgeon uses a drill 1202 to insert insertable item 1802 into real-world bone 1208. However, in other examples, the surgeon may use other types of insertable tools (e.g., an impactor) to insert insertable item 1802 into real-world bone 1208.

As shown in the example of FIG. 11, MR scene 1100 includes a first virtual guide 1210 and a second virtual guide 1212. Virtual guide 1210 and virtual guide 1212 are virtual objects designed to assist the surgeon with the task of aligning insertable item 1802 1802 with a planned insertion axis. In some examples, MR scene 1100 may include virtual guides 1210, 1212.

Virtual guide 1210 corresponds to anterior/posterior angles (e.g., the retroversion). Virtual guide 1212 corresponds to superior/inferior angles (e.g., the inclination). Virtual guide 1210 includes a target angle marker 1214 and virtual guide 1212 includes a target angle marker 1216. Target angle marker 1214 is located on virtual guide 1210 at a position corresponding to an anterior/posterior angle of the planned insertion axis. Similarly, target angle marker 1216 is located on virtual guide 1212 at a position corresponding to a superior/inferior angle of the planned insertion axis. Surgical assistance system 100 may determine the positions of target angle marker 1214 and target angle marker 1216 based on information in a surgical plan developed during a preoperative phase of the surgical procedure. In some examples, surgical assistance system 100 may always position target angle marker 1214 and target angle marker 1216 in the center of virtual guide 1210 and virtual guide 1212, respectively.

Furthermore, in the example of FIG. 11, virtual guide 1210 includes a current angle marker 1218 and virtual guide 1212 includes a current angle marker 1220. Current angle marker 1218 is located on virtual guide 1210 at a position corresponding to an anterior/posterior angle of the real-world current insertion axis of insertable item 1802. Similarly, current angle marker 1220 is located on virtual guide 1212 at a position corresponding to a superior/inferior angle of the real-world current insertion axis of insertable item 1802.

Surgical assistance system 100 may track the position of insertable item 1802 and/or drill 1202 as insertable item 1802 and drill 1202 move. For instance, surgical assistance system 100 may track the position of a marker attached to a physical tracking tool fitted on insertable item 1802, as described elsewhere in this disclosure.

Furthermore, surgical assistance system 100 may update the positions of current angle marker 1218 and current angle marker 1220 in response to changes in the position of insertable item 1802. For example, if the surgeon moves drill 1202 in an inferior direction, surgical assistance system 100 may update current angle marker 1220 to a position corresponding to a more inferior angle.

By moving drill 1202 in such a way that current angle marker 1218 is aligned with target angle marker 1214 in virtual guide 1210 and current angle marker 1220 is aligned with target angle marker 1216 in virtual guide 1212, the surgeon may align insertable item 1802 with the planned insertion axis. Thus, in this way, the surgeon may use virtual guide 1210 and virtual guide 1212 to help ensure that the surgeon is inserting insertable item 1802 into bone 1208 at the planned insertion angle.

In some examples, surgical assistance system 100 may also assist the surgeon with positioning insertable item 1802 at a planned insertion point. For example, surgical assistance system 100 may determine a current insertion point as a point at which a lengthwise axis of insertable item 1802 intersects a surface of bone 1208. Surgical assistance system 100 may include, in MR scene 1100, a virtual current insertion point object 1222 that indicates the current insertion point. Surgical assistance system 100 may update a position of virtual current insertion point object 1222 as the surgeon moves insertable item 1802. Additionally, in some examples, surgical assistance system 100 may include, in MR scene 1100, a virtual target insertion point object that indicates a target insertion point for insertable item 1802. Surgical assistance system 100 may determine the planned insertion point based on data stored in a surgical plan for the surgical procedure.

The following numbered clauses may illustrate one or more aspects of the disclosure:

Clause 1A: A method for registration, comprising: prompting a user wearing an XR headset to direct their gaze toward a first physical registration region of a physical object that corresponds to a first virtual registration region of a virtual model of the physical object; providing the user via the XR headset a visual cue in the vicinity of the first physical registration region, the visual cue indicating to the user a progress toward registration of the first physical registration region with the first virtual registration region.

Clause 2A: The method of clause 1A, wherein the first physical registration region is a first physical registration point or spot and the first virtual registration region is a first virtual registration point.

Clause 3A: The method of clause 1A, wherein providing the user the visual cue in the vicinity of the first physical registration region includes providing the visual cue within a predetermined angle of the first physical registration region, the predetermined angle being relative to a viewpoint of the user.

Clause 4A: The method of clause 1A, further comprising the user touching a registration instrument to the first physical registration region, the progress toward registration being timed relative to the touching of the registration instrument to the first physical registration region.

Clause 5A: The method of clause 4A, wherein the touching the registration instrument to the first physical registration region includes touching a tip of the registration instrument to the first physical registration region.

Clause 6A: The method of clause 5A, wherein the touching is continuous.

Clause 7A: The method of clause 5A, wherein the registration instrument includes a marker configured to be optically tracked by the XR headset.

Clause 8A: The method of clause 1A, wherein providing the visual cue includes providing visual cue graphics that generally surround the first physical registration region.

Clause 9A: The method of clause 8A, wherein the visual cue graphics include an arc.

Clause 10A: A computing system comprising a mixed reality (MR) visualization device; and one or more processors implemented in circuitry, the one or more processors configured to perform the method of any combination of clauses 1A-9A.

Clause 1B: A method for registration, the method comprising: prompting a user wearing a visualization device to direct their gaze toward a physical registration region of a physical object that corresponds to a virtual registration region of a virtual model of the physical object; providing the user, via a user interface of the visualization device, a visual cue in a vicinity of the physical registration region; performing a registration operation to register the physical registration region with the virtual registration region; and while performing the registration operation, changing the visual cue over time to indicate to the user a progress toward registration of the physical registration region with the virtual registration region.

Clause 2B: The method of clause 1B, wherein the physical object comprises a portion of an anatomy and the virtual model comprises a virtual model of the portion of an anatomy.

Clause 3B: The method of clause 1B, wherein the physical object comprises a surgical instrument and the virtual model comprises a virtual model of the surgical instrument.

Clause 4B: The method of clause 1B or 2B, further comprising: obtaining the virtual model from a virtual surgical plan for an orthopedic joint repair surgical procedure to attach a prosthetic to an anatomy.

Clause 5B: The method of any of clauses 1B-4B, wherein the visual cue comprises a virtual icon generated by the visualization device, the method further comprising: overlaying the virtual icon within the physical registration region.

Clause 6B: The method of any of clauses 1B-5B, wherein the visual cue comprises a curve that at least partially surrounds a center of the physical registration region and a geometric center of the curve is within the physical registration region.

Clause 7B: The method of clause 6B, wherein changing the visual cue over time to indicate to the user the progress toward registration of the physical registration region with the virtual registration region comprises changing the visual cue over time without changing a positioning of the geometric center of the curve with respect to a center of the physical registration region.

Clause 8B: The method of any of clauses 1B-5B, wherein the visual cue comprises a circle, wherein the circle surrounds a center of the physical registration region and a center of the circle is within the physical registration region.

Clause 9B: The method of clause 8B, wherein changing the visual cue over time to indicate to the user the progress toward registration of the physical registration region with the virtual registration region comprises changing the visual cue over time without changing a positioning of the center of the circle with respect to a center of the physical registration region.

Clause 10B: The method of any of clauses 1B-9B, wherein changing the visual cue over time to indicate to the user a progress toward registration of the physical registration region with the virtual registration region comprises increasing an amount of a first color on the visual cue and decreasing an amount of a second color on the visual cue.

Clause 11B: The method of any of clauses 1B-10B, wherein the physical object comprises an anatomical object.

Clause 12B: The method of any of clauses 1B-10B, wherein the physical object comprises a surgical tool.

Clause 13B: The method of clause 12B, wherein the surgical tool comprises a plurality of planar faces, each planar face having a different graphical pattern.

Clause 14B: The method of clause 13B, further comprising: obtaining image data generated by one or more cameras of the visualization device, wherein the image data depicts a scene comprising one or more of the planar faces; determining coordinates of a plurality of points on the graphical patterns; and determining, based on the determined coordinates and one or more properties of the surgical tool, a position and/or an orientation for an insertable object.

Clause 15B: The method of any of clauses 1B-14B, further comprising: outputting, via the user interface of the visualization device, one or more icons related to a status of the registration operation; overlaying the one or more icons within the physical registration region.

Clause 16B: The method of any of clauses 1B-15B, wherein performing the registration operation comprises executing minimization algorithm.

Clause 17B: The method of any of clauses 1B-16B, wherein performing the registration operation comprises generating a transformation matrix that aligns the virtual model of the physical object to the physical object.

Clause 18B: The method of any of clauses 1B-17B, wherein the visualization device comprises a mixed reality visualization device.

Clause 19B: A method for registration, the method comprising: prompting a user wearing a visualization device to place a tip of a registration instrument at a location on a physical object in a physical registration region; after placement of the tip, identifying the registration instrument within the physical registration region; using a positioning of the registration instrument to identify the location on the physical object; based on the identified location, performing a registration operation to register a virtual model of the physical object with the physical object; providing the user, via a user interface of the visualization device, a visual cue in a vicinity of the physical registration region; and while performing the registration operation, changing the visual cue over time to indicate to the user a progress toward registration of the virtual model of the physical object with the physical object.

Clause 20B: The method of clause 19B, wherein the registration instrument comprises a plurality of planar faces, each planar face having a different graphical pattern.

Clause 21B: The method of clause 20B, wherein using the positioning of the registration instrument to identify the location on the physical object comprises using one or more of the plurality of planar faces to determine the positioning of the registration instrument.

Clause 22B: The method of any of clauses 19B-21B, wherein the physical object comprises a portion of an anatomy and the virtual model comprises a virtual model of the portion of an anatomy.

Clause 23B: The method of any of clauses 19B-22B, wherein the visual cue comprises a virtual icon generated by the visualization device, the method further comprising: overlaying the virtual icon within the physical registration region.

Clause 24B: The method of any of clauses 19B-23B, wherein the visual cue comprises a curve that at least partially surrounds a center of the physical registration region and a geometric center of the curve is within the physical registration region.

Clause 25B: The method of clause 24B, wherein changing the visual cue over time to indicate to the user the progress toward registration of the virtual model of the physical object with the physical object comprises changing the visual cue over time without changing a positioning of the geometric center of the curve with respect to a center of the physical registration region.

Clause 26B: The method of any of clauses 19B-25B, wherein the visual cue comprises an arc of a circle, wherein the arc partially surrounds a center of the physical registration region and a center of the circle is within the physical registration region.

Clause 27B: The method of clause 26B, wherein changing the visual cue over time to indicate to the user the progress toward registration of the virtual model of the physical object with the physical object comprises changing the visual cue over time without changing a positioning of the center of the circle with respect to a center of the physical registration region.

Clause 28B: The method of clause 26B or 27B, wherein changing the visual cue over time to indicate to the user a progress toward registration of the virtual model of the physical object with the physical object comprises increasing an amount of a first color on the visual cue and decreasing an amount of a second color on the visual cue.

Clause 29B: The method of any of clauses 19B-28B, further comprising: outputting, via the user interface of the visualization device, one or more icons related to a status of the registration instrument; and overlaying the one or more icons within the physical registration region.

Clause 30B: The method of clause 29B, wherein a color of at least one of the one or more icons indicates a visibility status of the registration instrument.

Clause 31B: A computing system comprising: a mixed reality (MR) visualization device; and one or more processors implemented in circuitry, the one or more processors configured to perform the method of any combination of clauses 1B-30B.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuity,” as used herein may refer to any of the foregoing structures or any other structure suitable for implementation of the techniques described herein.

Various examples have been described. These and other examples are within the scope of the following claims.

1-18. (canceled)

19. A method for registration, the method comprising:
prompting a user wearing a visualization device to place a tip of a registration instrument at a location on a physical object in a physical registration region;
after placement of the tip, identifying the registration instrument within the physical registration region;
using a positioning of the registration instrument to identify the location on the physical object;
based on the identified location, performing a registration operation to register a virtual model of the physical object with the physical object;
providing the user, via a user interface of the visualization device, a visual cue in a vicinity of the physical registration region; and
while performing the registration operation, changing the visual cue over time to indicate to the user a progress toward registration of the virtual model of the physical object with the physical object.

20. The method of claim 19, wherein the registration instrument comprises a plurality of planar faces, each planar face having a different graphical pattern.

21. The method of claim 20, wherein using the positioning of the registration instrument to identify the location on the physical object comprises using one or more of the plurality of planar faces to determine the positioning of the registration instrument.

22. The method of claim 19, wherein the physical object comprises a portion of an anatomy and the virtual model comprises a virtual model of the portion of the anatomy.

23. The method of claim 19, wherein the visual cue comprises a virtual icon generated by the visualization device, the method further comprising:
overlaying the virtual icon within the physical registration region.

24. The method of claim 19, wherein the visual cue comprises a curve that at least partially surrounds a center of the physical registration region and a geometric center of the curve is within the physical registration region.

25. The method of claim 24, wherein changing the visual cue over time to indicate to the user the progress toward registration of the virtual model of the physical object with the physical object comprises changing the visual cue over time without changing a positioning of the geometric center of the curve with respect to the center of the physical registration region.

26. The method of claim 19, wherein the visual cue comprises an arc of a circle, wherein the arc partially surrounds a center of the physical registration region and a center of the circle is within the physical registration region.

27. The method of claim 26, wherein changing the visual cue over time to indicate to the user the progress toward registration of the virtual model of the physical object with the physical object comprises changing the visual cue over time without changing a positioning of the center of the circle with respect to the center of the physical registration region.

28. The method of claim 26, wherein changing the visual cue over time to indicate to the user the progress toward registration of the virtual model of the physical object with the physical object comprises increasing an amount of a first color on the visual cue and decreasing an amount of a second color on the visual cue.

29. The method of claim 19, further comprising:
outputting, via the user interface of the visualization device, one or more icons related to a status of the registration instrument overlaid within the physical registration region.

30. The method of claim 29, wherein a color of at least one of the one or more icons indicates a visibility status of the registration instrument.

31. (canceled)

32. A mixed reality system comprising:
a mixed reality (MR) visualization device configured to be worn by a user;
a memory; and
processing circuitry coupled to the memory and in communication with the MR visualization device, wherein the processing circuitry is configured to:
prompt the user wearing the MR visualization device to place a tip of a registration instrument at a location on a physical object in a physical registration region;
after placement of the tip, identify the registration instrument within the physical registration region;
use a positioning of the registration instrument to identify the location on the physical object;
based on the identified location, perform a registration operation to register a virtual model of the physical object with the physical object;
provide the user, via a user interface of the MR visualization device, a visual cue in a vicinity of the physical registration region; and
while performing the registration operation, changing the visual cue over time to indicate to the user a progress toward registration of the virtual model of the physical object with the physical object.

33. The mixed reality system of claim 32, wherein the registration instrument comprises a plurality of planar faces, each planar face having a different graphical pattern, wherein to use the positioning of the registration instrument to identify the location on the physical object, the processing circuitry is configured to use one or more of the plurality of planar faces to determine the positioning of the registration instrument.

34. The mixed reality system of claim 32, wherein the physical object comprises a portion of an anatomy and the virtual model comprises a virtual model of the portion of the anatomy.

35. The mixed reality system of claim 32, wherein the visual cue comprises a virtual icon generated by the MR visualization device, wherein the processing circuitry is further configured to:
overlay the virtual icon within the physical registration region.

36. The mixed reality system of claim 32, wherein the visual cue comprises a curve that at least partially surrounds a center of the physical registration region and a geometric center of the curve is within the physical registration region, and wherein to change the visual cue over time to indicate to the user the progress toward registration of the virtual model of the physical object with the physical object, the processing circuitry is configured to change the visual cue over time without changing a positioning of the geometric center of the curve with respect to the center of the physical registration region.

37. The mixed reality system of claim 32, wherein the visual cue comprises an arc of a circle, wherein the arc partially surrounds a center of the physical registration region and a center of the circle is within the physical registration region.

38. The mixed reality system of claim 37, wherein to change the visual cue over time to indicate to the user the progress toward registration of the virtual model of the physical object with the physical object, the processing circuitry is configured to change the visual cue over time without changing a positioning of the center of the circle with respect to the center of the physical registration region.

39. The mixed reality system of claim 37, wherein to change the visual cue over time to indicate to the user the progress toward registration of the virtual model of the physical object with the physical object, the processing circuitry is configured to increase an amount of a first color on the visual cue and decreasing an amount of a second color on the visual cue.
